(19) United States
(12) Patent Application Publication
(10) Pub. No.: US 2007/0225779 A1
(43) Pub. Date: Sep. 27, 2007

(54) TREATMENT OF VITILIGO BY MICROPORE DELIVERY OF CELLS

(75) Inventors: Basil M. Hantash, East Palo Alto, CA (US); George Frangineas, Fremont, CA (US); Leonard C. DeBenedictis, Palo Alto, CA (US)

Correspondence Address: RELIANT / FENWICK;c/o FENWICK & WEST, LLP, 801 CALIFORNIA STREET, MOUNTAIN VIEW, CA, 94041, US

(73) Assignee: Reliant Technologies, Inc., Mountain View, CA (US)

(21) Appl. No.: 11/753,472

(22) Filed: May 24, 2007

Related U.S. Application Data
(63) Continuation-in-part of application No. 11/370,657, filed on Mar. 7, 2006.

(51) Int. Cl. A61N 5/067; A61K 51/00

(52) U.S. Cl. 607/89; 424/1.17; 424/1.41; 424/423; 604/290

(57) ABSTRACT

Methods, compositions and apparatus for restoring pigmentation to skin that has suffered pigment loss are described. The methods include creating a plurality of spaced-apart microchannels or voids in the skin and depositing into the micropore channels or voids a composition comprising at least one cell capable of producing melanin, a growth factor, and, optionally, a scaffolding material, a differentiation factor, a proliferation factor, and/or a pigment. Alternatively, a composition comprising a pigment can be deposited into the micropore channels or voids to restore pigmentation to the skin.

### CROSS-REFERENCE TO RELATED APPLICATION

This application is a continuation-in-part of and claims priority to pending U.S. patent application Ser. No. 11/370,657, “Treatment of Alopecia By Micropore Delivery Of Stem Cells” by Basil M. Hantash et al., filed Mar. 7, 2006, the disclosure of which is incorporated herein by reference in its entirety.

### TECHNICAL FIELD OF THE INVENTION

The present invention relates in general to methods of treating vitiligo. The invention relates in particular to growing melanin from cells implanted in tissue in which loss of melanin has taken place.

### INTRODUCTION

Vitiligo, also known as leucoderma or achromia, is a common skin disorder in which the skin loses its normal pigmentation. It is a common problem in men and women worldwide, affecting about 2% of the world's population. Vitiligo results in white spots on the skin lacking pigmentation due to a lack of melanin, and in many cases is thought to occur when the subject's immune cells attack and kill melanocytes in the subject's skin. Hormonal conditions and genetic predisposition are also thought to play a role in some cases. As melanocytes are the pigment-producing cells in human skin, a decrease in the number or activity of melanocytes results in hypopigmentation of the skin. Vitiligo often starts as a single spot or a few spots which gradually grow in size and number. Often the spots will present in a symmetrical pattern on both sides of the body. Vitiligo can be associated with other conditions, including alopecia areata, premature graying of the hair, lichen planus, lichen sclerosis, psoriasis, halo naevus, and ichthyosis. Vitiligo can also be associated with systemic diseases, including thyroid disorders, systemic lupus erythematosus, pernicious anemia, Addison's Disease, collagen disease, Grave's Disease, and diabetes mellitus. Vitiligo has a significant impact on quality of life, mainly by causing emotional trauma and diminishing self-esteem.

Currently, only a few treatment options exist for vitiligo. These include topical tacrolimus, pimecrolimus, corticosteroids, and monobenzone, as well as phototherapy with ultraviolet radiation, including psoralen and long-wave ultraviolet radiation (PUVA) and narrowband ultraviolet B (UVB) radiation. The use of topical immune modulants (tacrolimus, pimecrolimus, corticosteroids) often provides little improvement, even after years of therapy. Depigmentation of the normal surrounding skin using monobenzone is not an acceptable solution for many subjects. The use of ultraviolet light to stimulate melanin production while suppressing the autoimmune destruction of melanocytes is limited due to side effects such as sunburn reactions, blisters, and concerns about increased risk of skin cancer due to the need for prolonged and repeated treatments.

Surgical treatments, including suction blister grafting, split-thickness skin grafting, punch grafting, follicular grafting, cultured melanocyte transplantation, and non-cultured melanocyte transplantation, have been used to treat of vitiligo, but these treatments are very difficult and have varying rates of success, and so have not been widely adopted to date. Also, techniques such as punch grafting often result in a “cobblestoned” or uneven distribution of melanin in the treated area, as well as scarring.

A method of restoring pigmentation such as promoting melanin production in a non-invasive or minimally invasive manner that results in a consistent distribution of pigmentation in the treated skin with little or no scarring is therefore desirable. In humans, the pigment primarily responsible for skin color is melanin. One possible method would be to introduce cells capable of producing melanin into the skin, where the cells would become part of the skin and begin producing melanin, serving as an on-going source of melanin in the skin. The density and type of cells capable of producing melanin introduced into the skin could be controlled and/or augmented with a pigment so as to control the level of pigmentation and color of the skin, in order to best match the existing normal skin of the subject and create a natural-looking color pattern in the skin. A non-invasive or minimally invasive procedure could be repeated as necessary to obtain the desired level of pigmentation and color of the skin, to “touch up” or expand a region of treatment if needed, or to repeatedly re-introduce cells capable of producing melanin if the condition re-emerged following treatment.

Cells capable of producing melanin include melanocytes, melanophores, melanoblasts, and stem cells. In humans, dermal melanin is produced by melanocytes, which are found in the basal layer of the epidermis. Melanocytes originate in the neural crest and migrate to the basal layer of the epidermis and hair matricies, among other locations, unlike the surrounding basal skin cells. Typically, melanocytes constitute between about 5% and about 10% of the basal layer skin cells, i.e., one cell in about every 10 to 20 basal skin cells is a melanocyte. On this basis, for every square millimeter of skin, there are between about 1000 and about 2000 melanocytes. The average diameter of a melanocyte is approximately 7 micrometers (μm), while the average diameter of epidermal skin cells (keratinocytes) range from about 11 μm to about 13 μm. Typically, each melanocyte supplies melanin to approximately 30 nearby keratinocytes via its dendrites. Humans generally possess a similar concentration of melanocytes in their skin; it is the level of activity of the melanocytes that gives rise to differences in skin color between individuals. For example, lighter-skinned individuals generally have low basal levels of melanin production, and exposure to UV radiation generally causes increased melanin production.

In melanocytes, melanin is produced in organelles known as melanosomes by the process of melanogenesis. As melanin is an aggregate of smaller compound molecules, there are a number of different types of melanin. The two major forms of melanin are eumelanin, which is brown to black in color, and pheomelanin, which is yellow to red in color. The regulation of the production of eumelanin versus pheomelanin involves the interaction of the melanocortin 1 receptor (MC1R) on the surface of the melanocyte with melanocyte stimulating hormone (MSH) or with the agouti signaling protein. The binding of MSH to MC1R results in the formation of eumelanin while the binding of the agouti protein to MC1R leads to the production of pheomelanin.

Other cell types capable of producing melanin include melanophores, melanoblasts, and stem cells. Melanophores are pigment-producing cells most commonly found in amphibians and reptiles that contain melanin. Melanoblasts are cells that give rise to either melanocytes or melanophores. Stem cells are the body's “master” cells with the ability to grow into any one of the body's more than 200 cell types. Stem cells have recently received a significant amount of attention due to their potential to regenerate tissue and organs. For example, stem cells isolated from the hair bulge region of the follicle explanted into nude mouse skin have given rise to hair follicles and sebaceous glands in animal models. To date, no method has been developed in order to utilize stem cells in humans to produce melanin to treat problems such as vitiligo.

Stem cells can be somewhat difficult to work with. It can be difficult to isolate these rare cells from the donor. Although methods exist for expanding the stem cell in vitro, each passage of stem cells during tissue culture diminishes the odds that multipotential differentiation is preserved. Furthermore, for some types of stem cells, expansion may not be feasible as some types of stem cells lose their ability to differentiate after the first passage. To avoid issues of immune-dependent rejection, the recipient can serve as the donor.

One difficulty in using cells capable of producing melanin to treat conditions such as vitiligo lies in finding an effective method of depositing the cells capable of producing melanin into the skin of the subject. Such a method must be relatively painless and preferably capable of being implemented over relatively large areas, for example, one-hundred square centimeters (cm²) or more. One possible approach could involve an instrument-dependent, “cookie-cutter” approach of mechanically perforating or cutting skin to provide channels for receiving cells capable of producing melanin. Such approaches can be painful, tedious, time-consuming, and can pose significant wound healing difficulties.

### SUMMARY OF THE INVENTION

The present invention is directed to apparatus, methods and compositions for restoring pigmentation to a subject that has suffered a loss of pigmentation.

In one aspect, the present invention provides a method of treating or preventing pigmentation loss in the skin of a subject in need thereof, the method comprising irradiating skin with laser irradiation to form a plurality of micropore channels wherein the micropore channels extend into the basal layer of the epidermis; and implanting a composition into the micropore channel, wherein the composition comprises at least one cell capable of producing melanin, and a growth media.

In another aspect, the present invention provides an apparatus for treating or preventing pigmentation loss in the skin of a subject in need thereof, the apparatus comprising a handpiece movable over skin wherein the handpiece is arranged to receive an optical beam and focus the optical beam at a plurality of spaced-apart locations on the skin thereby creating a plurality of voids in the skin for the deposition of a composition, wherein the composition comprises at least one cell capable of producing melanin and a growth media. In yet another aspect, the present invention provides compositions for providing pigmentation and/or promoting melanin production in the skin. The compositions can comprise a pigment such as a tattoo ink. Alternatively, the compositions can comprise a pigment in combination with at least one cell capable of producing melanin and a growth factor. The compositions can be delivered into the skin by depositing the compositions into one or more micropore channels or voids in the skin.

### BRIEF DESCRIPTION OF THE DRAWINGS

The accompanying drawings, which are incorporated in and constitute a part of the specification, schematically illustrate a preferred embodiment of the present invention, and together with the general description given above and the detailed description of the preferred embodiment given below, serve to explain principles of the present invention.

FIG. 1 is a micrograph of a section of human skin immediately after irradiation with laser radiation having parameters in accordance with the method of the present invention, the irradiated skin including a plurality of voids extending through the stratum corneum and the epidermis into the dermis, the voids being surrounded by regions of coagulated dermal tissue with viable tissue between the regions of coagulated tissue surrounding the voids.

FIG. 2 is a micrograph similar to the micrograph of FIG. 1 but having a lower magnification and depicting detail of the voids extending through the stratum corneum.

FIG. 3 is a micrograph of a section of human skin 48 hours after irradiation with laser radiation having parameters in accordance with the method of FIG. 1.

FIG. 4 is a micrograph of a section of human skin one week after irradiation with laser radiation having parameters in accordance with the method of FIG. 1.

FIG. 5 is a micrograph of a section of human skin one month after irradiation with laser radiation having parameters in accordance with the method of FIG. 1.

FIG. 6 is a graph schematically illustrating trend curves for maximum lesion or treatment zone with (void width plus coagulated tissue width) as a function of lesion or zone depth in the method of the present invention, for 5 mJ, 10 mJ, and 20 mJ pulses.

FIG. 7 is a graph schematically illustrating trend curves for maximum void width as a function of lesion or zone depth in the method of the present invention, for 5 mJ, 10 mJ, and 20 mJ pulses.

FIGS. 8A, 8B, and 8C are graphs schematically illustrating estimated width as a function of lesion or zone depth for lesions and voids with dimensions derived from micrographs of treatment sites in accordance with the present invention, for respectively 5 mJ, 10 mJ, and 20 mJ pulses.

FIG. 9A is a front elevation view schematically illustrating one example of apparatus suitable for irradiating skin according to the method of the present invention, the apparatus including a multi-faceted scanning wheel for scanning a pulsed, collimated laser beam and a wide field lens for focusing the scanned laser beam onto skin to sequentially ablate tissue and create the cauterized voids of the inventive method.

FIG. 9B is a front elevation view schematically illustrating further detail of beam focusing in the apparatus of FIG. 9A.

FIG. 9C is a side elevation view schematically illustrating still further detail of beam focusing in the apparatus of 9A.

FIG. 10 schematically illustrates detail of the scanning wheel of FIGS. 9A-C.

FIG. 11 schematically illustrates one example of a handpiece including the apparatus of FIGS. 9A-C, the handpiece including a removable tip connectable to a vacuum pump for exhausting smoke and ablation debris from the path of the laser beam.

### DETAILED DESCRIPTION OF THE INVENTION

Unless otherwise stated, the following terms used in this application, including the specification and claims, have the definitions given below. It must be noted that, as used in the specification and the appended claims, the singular forms “a,” “an” and “the” include plural referents unless the context clearly dictates otherwise. The practice of the present invention will employ, unless otherwise indicated, conventional methods of preparative and analytical methods of chromatography, protein chemistry, biochemistry, recombinant DNA techniques and pharmacology, within the skill of the art.

The present invention provides methods, compositions and apparatus for the prevention and/or treatment of pigmentation loss in a subject. The pigmentation loss can occur in the skin of the subject. The pigmentation loss can be due to vitiligo. The compositions of the invention can comprise at least one cell capable of producing melanin and a growth media. The compositions can optionally include a scaffolding material, a proliferation factor, a differentiation factor, and/or a pigment. The compositions can be delivered into the skin by depositing the compositions into one or more micropore channels or voids in the skin.

Also included are methods, compositions and apparatus for the delivery of compositions comprising pigment alone or in combination with cells capable of producing melanin and a growth factor. The methods, compositions and apparatus can be used for a variety of purposes, including, but not limited to, prevention and/or treatment of pigmentation loss in a subject. The methods, compositions and apparatus can provide pigment alone or can provide pigment while promoting melanin production in the skin. The compositions can be delivered to treat hypopigmentation. The compositions can be delivered into skin to provide pigmentation and/or to promote melanin production in the skin. The compositions can comprise a pigment such as a tattoo ink. Alternatively, the compositions can comprise a tattoo ink in combination with at least one cell capable of producing melanin and a growth factor. The compositions can be delivered into the skin by depositing the compositions into one or more micropore channels or voids in the skin.

In another aspect of the invention, laser radiation is used to form the spaced-apart micropore channels or voids, thereby causing the micropore channels or voids to be surrounded with coagulated tissue immediately following the irradiation. There is viable tissue remaining between the micropore channels or voids. The coagulated tissue is under tension resulting from collagen shrinkage by heat generated during the coagulation and/or ablation process. The tension in the coagulated tissue shrinks the micropore channels or voids. The cells capable of producing melanin, a growth factor or factors, and optionally a scaffold, a proliferation factor, a differentiating factor, and/or a pigment are deposited into the micropore channels or voids. The cells capable of producing melanin become part of the skin, over time producing melanin which can be taken up by the surrounding keratinocytes. Depending upon factors such as the level of activity of previously deposited cells and/or pigment or the desired level of pigmentation or coloration desired, the cells capable of producing melanin can be encouraged or discouraged from proliferating. A healing process completely replaces the coagulated tissue with new tissue after a period of about one month, while the cells capable of producing melanin and, optionally, a pigment, remain present in the skin, producing melanin in the skin and imparting pigmentation to the skin.

In another aspect, the present invention provides an apparatus for treating or preventing pigmentation loss in the skin of a subject in need thereof, wherein the apparatus comprises a handpiece movable over skin, and wherein the handpiece is arranged to receive an optical beam and focus the optical beam at a plurality of spaced-apart locations on the skin, thereby creating a plurality of micropore channels or voids in the skin for the deposition of a composition, wherein the composition comprises at least one cell capable of producing melanin, a growth media, and, optionally, a scaffold, a proliferation factor, a differentiation factor, and/or a pigment.

The micropore channels or voids are created by exposing the skin to laser radiation in a manner so as to create a plurality of micropore channels or voids in the skin, wherein viable tissue separates the plurality of micropore channels or voids In one aspect, the compositions are implanted into one or more micropore channels or voids in the skin, wherein the micropore channels or voids are created using laser irradiation of the skin. In another aspect, the compositions are injected into one or more micropore channels or voids in the skin, wherein the micropore channels or voids are created using laser irradiation of the skin.

The micropore channels or voids can extend through the stratum corneum and the epidermis into the basal layer of the epidermis and are surrounded by regions of coagulated tissue. The micropore channels or voids can extend through the epidermis into the dermal-epidermal junction and are surrounded by regions of coagulated tissue. The micropore channels or voids can extend through the epidermis into the dermis and are surrounded by regions of coagulated tissue. In one example, the micropore channels or voids can be created using an electromagnetic radiation treatment that is delivered in a fractional manner, leaving viable tissue present between adjacent micropore channels or voids. The viable tissue remaining between the micropore channels and voids helps promote healing of the micropore channels or voids. Additionally, ablative electromagnetic radiation treatments delivered in a fractional manner can be optimized so as to produce voids of a desired depth and diameter. One method of producing such voids is by the use of C0₂ laser treatments delivered in a fractional manner.

The process of producing micropore channels or voids using electromagnetic energy delivered in a fractional manner can be used to create a regenerative environment in the skin. The creation of a regenerative environment in the skin by producing the voids using electromagnetic energy induces the surrounding untreated tissue to migrate into the void.

The creation of a regenerative environment in the skin by producing voids also stimulates one or more regenerative signals within the tissue. The regenerative signal can comprise a growth factor, a cytokine, and the like. The regenerative signal induces invagination of epidermal stem cells into the void. The regenerative signal initiates a cascade of regenerative signals with temporal equivalence to the regeneration signals of embryological skin.

The regenerative signal can mimic the embryological pigment induction signal. By mimicking the embryological pigment induction signal, the regenerative signal can promote the induction of pigment regeneration upon introduction of an exogenous stem cell.

The regeneration signal and/or the invagination of epidermal stem cells into the void can promote the attachment, development and/or proliferation of an exogenous cell, such as, for example, a stem cell placed into the void. The exogenous cell can be a melanocyte. The exogenous cell can be a stem cell from an autologous source. The exogenous cell can be a stem cell from an allogeneic source. The stem cell can be an epidermal stem cell, a dermal papilla stem cell, a stem cell that is mesenchymal in origin, a stem cell that is embryonic in origin, a stem cell that is ectodermal in origin, or a combination thereof.

Stem cells useful for producing melanin can be derived from a mammal, such as a human, mouse, rat, pig, sheep, goat, or non-human primate. Stem cells are unspecialized cells capable of extensive proliferation. Stem cells are pluripotent and are believed to have the capacity to differentiate into most cell types in the body, including neural cells, muscle cells, blood cells, epithelial cells, skin cells, and hair cells. Further, stem cells are capable of ongoing proliferation in vitro without differentiating. As they divide, they retain a normal karyotype, and they retain the capacity to differentiate to produce adult cell types.

Aspects according to the invention include methods for delivering at least one cell capable of producing melanin. The at least one cell capable of producing melanin can include a melanocyte, a melanoblast, a melanophore, a stem cell, and combinations thereof, to the subject in need of therapy. The at least one cell capable of producing melanin can be endogenous or exogenous. The at least one cell capable of producing melanin can be a cultured cell. The cell capable of producing melanin can be a human cell.

The at least one cell capable of producing melanin can be a stem cell. The stem cell can be derived from an epidermal, adult, fetal, umbilical cord blood or embryonic source. The stem cell can be derived from, for example, the hair follicle bulge and dermal papilla, epidermal layer of skin, adipose tissue, bone marrow, or peripheral blood of an individual, Alternatively, the stem cell can be derived from an embryonic stem cell isolated from the inner cell mass of a pre-implantation embryo. The stem cell can also be derived from umbilical cord blood or from fetal tissue. The stem cell can be a melanocyte stem cell.

The at least one cell capable of producing melanin can be chosen to reflect the skin color of the subject. As the level and type of melanin produced can vary from individual to individual based on racial and genetic factors, it is desirable to try to account for these factors to obtain the most natural looking treatment possible. A catalog of several skin color types can be used, wherein the skin color types can include light, medium and dark types, as well as different color ranges based on the proportions of eumelanin and pheomelanin produced by an individual's naturally occurring melanocytes. Mixtures of different types of cells capable of producing melanin can also be used to approximate the natural skin color of the subject. For example, a number of types of cultured human melanocytes from sources such as adult skin and foreskin are commercially available from PromoCell (Heidelberg, Germany). Different color types of human melanocytes (light, medium and dark) are commercially available from ScienCell (San Diego, Calif., USA).Optionally, the at least one cell can be combined with a scaffolding material, a bioactive agent such as a growth factor, a differentiation factor, a proliferation factor, other bioactive agents, a pigment, and/or a carrier solution.

The stem cell can be derived from the skeletal muscle, adipose tissue, bone marrow, skin, or other tissue samples, or the cells may be cultured, expanded, combined or manipulated before use. One cell type or a combination of cell types can be used. Optionally, the stem cell can be combined with a scaffolding material, a bioactive agent such as a growth factor, a differentiation factor, a proliferation factor, other bioactive agents, a pigment, and/or a carrier solution.

Stem cells suitable for implantation in the present invention include stem cells from embryonic, fetal, umbilical cord blood, and adult stem cells. Typically, these stem cells differentiate to form different types of cells, or, they can be converted to a wide variety of immunologically neutral cells that have been programmed to function as undifferentiated pluripotent cells. The cells can be genetically engineered or non-engineered, and mixtures of such cells also can be used. The cell can be modified such that it is surface antigen negative for CD44, CD45, and HLA Class I and II. The cell can also be surface antigen negative for CD34, Muc18, Stro-1, HLA-class-I and can be positive for oct¾ mRNA, and hTRT mRNA. In particular, the cell can be surface antigen negative for CD31, CD34, CD36, CD38, CD45, CD50, CD62E and CD62P, HLA-DR, Muc18, STRO-1, cKit, Tie/Tek, CD44, HLA-class I and 2-microglobulin and is positive for CD10, CD13, CD49b, CD49e, CDw90, Flk1, EGF-R, TGF-R1 and TGF-R2, BMP-R1A, PDGF-R1 and the like. The cells with modified surface antigens can be useful for modulating the immunological response, such as, for example, reducing the immunogenicity of the transplanted cells.

Typically, the tissue is harvested from, for example, adipose tissue, bone marrow, blood, skin, or other tissues where melanocytes, melanoblasts or adult stem cells may be found, fetal tissue, umbilical cord blood, or embryos where embryonic stem cells are found. Further, cells can be obtained from donor tissue, such as donor skin or scalp, by dissociation of individual cells from the connecting extracellular matrix of the tissue. Tissue can be removed using a sterile procedure, and the cells can be dissociated using any method known in the art including treatment with enzymes such as trypsin, collagenase, and the like, or by using physical methods of dissociation such as with a blunt instrument. For example, adipose tissue is readily accessible and abundant in most individuals and can be harvested by liposuction. Various liposuction techniques exist, including ultrasonic-assisted liposuction (“UAL”), laser-assisted liposuction, and traditional suction-assisted liposuction (“SAL”), where fat is removed with the assistance of a vacuum created by either a mechanical source or a syringe. Each of the foregoing liposuction techniques can be used in conjunction with tumescent solution. Liposuction procedures that use a tumescent solution generally involve pre-operative infiltration of subcutaneous adipose tissue with large volumes of dilute anesthetic solutions. Adipose may also be harvested during panniculectomy or abdominoplasty procedures.

Another advantage of using adipose tissue as a source of adult stem cells is that, due to the abundance of stem cells in adipose tissue, stem cell harvest, isolation, genetic manipulation and/or growth-factor based differentiation may be accomplished peri-operatively. Thus, depending on the number of cells required for implantation, it may not be necessary for the subject to submit to the liposuction procedure on one day and the cell implantation on a subsequent day. The procedures can be performed sequentially within minutes or tens of minutes of one another.

In another aspect, the stem cells can be melanocyte stem cells isolated from tissue of an adult mammal, preferably a human. The cells include but are not limited to, melanocyte stem cells responsible for producing melanocytes that provide pigmentation. Different stem cells can be isolated from other cells by means known in the art. Melanocytes can be readily identified from other cells. For example, melanocytes contain microphthalmia transcription factor. Although the multi-potent stem cell that gives rise to a melanocyte is exemplified herein, the methods of ex vivo propagation described herein can be applied to any stem cell whether it be muti-potent, pluripotent, or a unique progenitor subtype, such as a stem cell that produces only sebaceous glands and not, for example, sweat glands.

The somatic melanocyte stem cells act as precursor cells, which produce daughter cells that mature into differentiated melanocytes. The melanocytes can be isolated from the individual in need of therapy, or from another individual. Somatic melanocyte stem cells may be immune-privileged, so the graft versus host disease after allogenic transplant may be minimal or non-existent. Melanocyte stem cells can be administered by any known means, for example, intravenous injection, or injection directly into the appropriate tissue, such as hypopigmented skin.

Alternatively, bone marrow may be harvested for adult stem cells. Bone marrow is a complex tissue comprised of two distinct populations of stem cells, namely hematopoietic stem cells and mesenchymal stem cells. Hematopoietic stem cells give rise to components of the blood and immune systems while mesenchymal stem cells give rise to varied cells, including osteoblasts, chondrocytes, adipocytes, fibroblasts, smooth muscle cells, and myoblasts. Cells, such as fibroblasts, reticulocytes, adipocytes and endothelial cells, form a connective tissue network called “stroma.” Cells from the stroma regulate morphologically the differentiation of hematopoietic cells through direct interaction via cell surface proteins and the secretion of growth factors.

In yet another embodiment, adult stem cells may be derived from peripheral blood. Human blood has circulating adult progenitor cells that can be capable of differentiating into hair cells in response to platelet derived growth factor (PDGF-BB) treatment. Thus, in one aspect of the present invention, a blood draw is contemplated. Since progenitor cell populations are present in blood in very low percentages, the cells are expanded in culture following growth factor-induced differentiation and selection. Alternatively, the subjects may be systemically treated with agents, such as granulocyte-colony stimulating factor (G-CSF), granulocyte monocyte colony-stimulating factor (GM-CSF), or the like.

As used herein, the term “growth media” refers to a composition containing a protein, peptide or other molecule having a growth, or trophic effect on cells capable of producing melanin. Growth media that can be used include any trophic factor that allows or encourages cells capable of producing melanin to grow, integrate into the skin, produce melanin, or proliferate, including any molecule that binds to a receptor on the surface of the cell to exert a trophic, or growth-inducing effect on the cell. The growth media can be a melanocyte growth media. The melanocyte media can be formulated quantitatively and qualitatively to provide a defined and optimally balanced nutritional environment that promotes growth or growth and proliferation of normal human melanocytes in vitro. Factors which can be present in the melanocyte growth media include, but are not limited to, basic fibroblast growth factor (bFGF), bovine pituitary extract (BPE), fetal bovine serum, hydrocortisone, insulin, phenol red, phorbol myristate acetate (PMA), epidermal growth factor (EGF), transferrin, epinephrine, calcium chloride, penicillin, streptomycin, gentamycin, Amphotericin B, mixtures thereof, and the like. The growth media can be a sterile liquid media that contains essential and non-essential amino acids, vitamins, organic and inorganic compounds, hormones, growth factors, trace minerals, and a low concentration of fetal bovine serum or an animal-free substitute. The media can be HEPES and bicarbonate buffered at a pH of about 7.4. An example of a melanocyte media contains 500 ml of basal medium, 5 ml of melanocyte growth supplement, and 2.5 ml of fetal bovine serum. Alternatively, several formulations growth media, such as melanocyte basal medium, melanocyte growth medium and melanocyte growth supplements, are commercially available from, for example, PromoCell, ScienCell and Provitro GmbH (Berlin, Germany).

Cells can be isolated from harvested tissue. In general, methods of isolation of cells include not only harvesting a tissue specimen, but also processing the specimen so that the cells contained therein are substantially dissociated into single cells rather than grouped as cell clusters. Dissociating the cells into single cell components can be accomplished by any method known in the art; e.g., by mechanical (filtering) or enzymatic means. Further, the isolating step includes combining the cell-containing specimen with a cell culture medium comprising factors that stimulate cell growth without differentiation. Next, the specimen-medium mixture is cultured for a few up to many cell passages.

Appropriate culture medium is described in the art. For example, stem cells can be cultured in serum free DMEM/high-glucose and F12 media (mixed 1:1), and supplemented with N2 and B27 solutions and growth factors. Growth factors such as EGF, IGF-1, and bFGF have been demonstrated to augment sphere formation in culture. In vitro, stem cells often show a distinct proliferation potential for forming spheres. Thus, the identification and isolation of spheres can aid in the process of isolating stem cells from mature tissue for use in making differentiated cells. The growth medium for cultured stem cells can contain one or more or any combination of growth factors, provided that the stem cells do not differentiate. To induce the cells (and the cells of the spheres) to differentiate, the medium can be exchanged for medium lacking growth factors. For example, the medium can be serum-free Dulbecco's modified Eagle's medium (DMEM)/high glucose and F12 media (mixed about 1:1) supplemented with N2 and B27 solutions. Another example of a serum-free media includes DMEM/low glucose supplemented with about 1 ng/ml to about 100 ng/ml, about 4 ng/ml to about 40 ng/ml, or about 40 ng/nl of basic fibroblast growth factor (bFGF). The media can be used in the absence of a feeder layer, but in the presence of a matrix coated tissue culture dish. The matrix can be selected from fibronectin, collagen, laminin, and combinations thereof. Equivalent alternative media and nutrients can also be used. Culture conditions can be optimized using methods known in the art.

Cells can be cultured in suspension or on a fixed substrate. For example, the cells can be grown on a hydrogel, such as a peptide hydrogel, as described below. Alternatively, the cells can be propagated on tissue culture plates or in suspension cultures. Cell suspensions can be seeded in any receptacle capable of sustaining cells, particularly culture flasks, cultures plates, or roller bottles, more particularly in small culture flasks such as 25 cm² cultures flasks. The cells capable of producing can be grown on tissue culture plates, and can be cultured at high cell density to promote the suppression of asymmetric cell kinetics.

Conditions for culturing should be close to physiological conditions. The pH of the culture medium should be close to physiological pH, preferably between pH 6-8, more preferably between about pH 7 to 7.8, with pH 7.4 being most preferred. Physiological temperatures range between about 30° C. to 40° C. Cells are preferably cultured at temperatures between about 32° C. to about 38° C., and more preferably between about 35° C. to about 37° C. Cells are preferably cultured for 3-30 days, preferably at least about 7 days, more preferably at least 10 days, still more preferably at least about 14 days. Cells can be cultured substantially longer. They can also be frozen using known methods such as cryopreservation, and thawed and used as needed.

Another embodiment provides for deriving clonal lines of somatic cells by limiting dilution plating or single cell sorting. Methods for deriving clonal cell lines are well known in the art.

The compositions can optionally comprise a scaffolding material. The scaffolding material can be used to weigh down the deposited material and/or to keep the deposited material in place during the healing process. The scaffolding material can be a natural or synthetic material. The scaffolding material can be a cellular scaffolding material. The scaffolding material can be a self-assembling material. The scaffolding material can comprise poly(lactic-co-glycolic acid) (PLGA), fibronectin, collagen 1, collagen 3, peptide hydrogels, carbon nanotubes, mixtures thereof, and the like.

The compositions can optionally comprise a differentiation factor. The differentiation factor can be one or more selected from the group consisting of nerve growth factor (NGF), platelet-derived growth factors (PDGF), thryotropin releasing hormone (TRH), transforming growth factor betas (TGFβs), insulin-like growth factor (IGF-1), combinations thereof, and the like.

Protocols for the identification of cells that differentiate into cells capable of producing melanin are known. The cells can be monitored for expression of cell-specific markers. For example, cells capable of producing melanin can be identified by the expression of Tyrosinase, Tyrp1, Dct, microphthalmia-associated transcription factor (Mitf), S, Kit, Sox10, Lef1, Mki67, or Ta1. Selection can be accomplished by fluorescence activated cell sorting (FACS), magnetic activated cell sorting (MACS), western blotting, or by other techniques known by those skilled in the art.

The changes that induce a stem cell to differentiate, such as into a melanocyte, involve altered biochemical pathways that lead to a specific phenotype. These alterations are a result of the expression of specific genes, and this expression pattern can be influenced by signals from the environment of the cell including cell-cell contact, oxygen content, nutrient availability, ligands that bind to receptors on the cells, temperature, and other factors.

Proteins that influence (e.g., promote or inhibit differentiation) the phenotype of cells capable of producing melanin include developmental regulators, cell cycle inhibitors, transcription factors and other regulatory proteins that act on stem cells. The phenotype of the cell includes the characteristics that distinguish it from other cell types. For example, the phenotype of a cell capable of producing melanin is distinct from the phenotype of a spiral ganglion cell.

Agents capable of causing stem cells to differentiate are referred to as differentiation factors. Differentiation factors can be, for example, small molecules, antibodies, peptides (e.g., peptide aptamers), antisense RNAs, small inhibitory RNAs (siRNAs), or ribozymes. Differentiation factors, such as small molecules, can modulate the activity of one or more of the proteins that influence cell phenotype by altering the activity of a growth factor or receptor, an enzyme, a transcription factor, or a cell-specific inhibitor. These molecules can change the binding affinity of a protein for another protein, or can bind in an active site of an enzyme or act as an agonist or antagonist of a ligand binding to a receptor. Some types of differentiation factors, such as small inhibitory RNAs (siRNAs), antisense RNAs, or ribozymes, can modify the expression pattern of genes that encode these proteins. Furthermore, the factors can be useful as therapeutic agents for treating hearing disorders or vestibular dysfunction.

A differentiation factor can cause a stem cell to differentiate, at least partially, into a cell capable of producing melanin. The differentiation factor can be a polypeptide, such as an aptamer or antibody; a nucleic acid, such as DNA or RNA; or a compound, such as a small molecule. For example, a factor is contacted with a stem cell, and the stem cell is determined to differentiate, at least partially, into a cell capable of producing melanin. The differentiation factor can be naturally occurring or synthetic, and can be obtained from a library, or identified by other methods.

A variety of methods can be utilized to determine that a stem cell has differentiated at least partially into a cell capable of producing melanin. For example, the cell can be examined for the expression of a cell marker gene. Gene markers for cells capable of producing melanin include Dct, Pax3, Tyr, S, Tyrp1 , Kit, microphthalmia-associated transcription factor (Mitf), Sox10, Lef1, Mki67, or Ta1. For example, melanocyte stem cells are Dct⁺, Pax3⁻, Tyr⁻, S⁻, Tyrp1⁻, Kit⁻, Mitf¹, Sox10⁻, Lef1⁻, Mki67⁻, and Ta1⁻. Melanoblasts and melanocytes are Dct⁻, Pax3⁺, Tyr⁺, S⁺, Tyrp1⁻, Kit⁺, Mitf⁺, Sox10⁺, Lef1⁺, Mki67⁺, and Ta1⁺. A pluripotent stem cell does not express these genes. A stem cell that propagates and produces a cell expressing one or more of these genes, has produced a cell capable of producing melanin, i.e., the stem cell has differentiated at least partially into a cell capable of producing melanin. A stem cell that has differentiated into a progenitor cell (a precursor of cells capable of producing melanin) expresses early marker genes. A progenitor cell can express one or more of these genes. The progenitor cells can be propagated in serum-free medium in the presence of growth factors. Removal of growth factors will induce the cells to differentiate further, such as into cells capable of producing melanin.

Identification of a cell or cell progenitor (e.g., a melanocyte or progenitor cell that differentiated from a stem cell) can be facilitated by the detection of expression of tissue-specific genes. Detection of gene expression can be by immunocytochemistry. Immunocytochemistry techniques involve the staining of cells or tissues using antibodies against the appropriate antigen. In this case, the appropriate antigen is the protein product of the tissue-specific gene expression. Although, in principle, a first antibody (i.e., the antibody that binds the antigen) can be labeled, it is more common (and improves the visualization) to use a second antibody directed against the first (e.g., an anti-IgG). This second antibody is conjugated either with fluorochromes, or appropriate enzymes for calorimetric reactions, or gold beads (for electron microscopy), or with the biotin-avidin system, so that the location of the primary antibody, and thus the antigen, can be recognized. The protein marker can also be detected by flow cytometry using antibodies against these antigens, or by Western blot analysis of cell extracts.

Tissue-specific gene expression can also be assayed by detection of RNA transcribed from the gene. RNA detection methods include reverse transcription coupled to polymerase chain reaction (RT-PCR), Northern blot analysis, and RNAse protection assays.

In some embodiments, a differentiation factor can be tested against stem cells that have been engineered to express a reporter gene that facilitates detection of cells converted into inner ear cells. These engineered stem cells make up a reporter cell line. A reporter gene is any gene whose expression may be assayed; such genes include, without limitation, green fluorescent protein (GFP), α-glucuronidase (GUS), luciferase, chloramphenicol transacetylase (CAT), horseradish peroxidase (HRP), alkaline phosphatase, acetylcholinesterase and β-galactosidase. Other optional fluorescent reporter genes include but are not limited to red fluorescent protein (RFP), cyan fluorescent protein (CFP) and blue fluorescent protein (BFP), or any paired combination thereof, provided the paired proteins fluoresce at distinguishable wavelengths.

A reporter gene can be under control of a promoter that is active in cells capable of producing melanin, including progenitor cells and cells at varying degrees of differentiation, but not in stem cells. Ideally, the promoter is stably upregulated in the differentiated cells or progenitor cells to allow assessment of the partially or fully differentiated phenotype (e.g., expression of the reporter gene and further identification of genes known to be expressed in the cell capable of producing melanin). In one exemplary embodiment, the luciferase gene is the reporter gene, which is under control of a promoter active in cells capable of producing melanin. By choosing a promoter expressed primarily in cells capable of producing melanin and in only a few other cell types, the partial or full conversion of the stem cells to cells capable of producing melanin will result in increased luminescent signal, whereas conversion of stem cells to most other cell types will not increase luciferase expression.

Depending upon factors such as the level of pigmentation desired following treatment, the density of the treatment, and the diameter of the micropore channels or voids, it may or may not be desirable to promote proliferation of the cells capable of producing melanin. The compositions can optionally comprise a proliferation factor. The proliferation factor can be selected from the group consisting of basic fibroblast growth factor (bFGF or FGF-2), hepatocyte growth factor/scatter factor (HGF/SF), macrophage colony stimulating factor (M-CSF), endothelin-1 (ET-1), melanocyte stimulating hormone (MSH), transforming growth factor-beta (TGFβ), bovine pituitary extract (BPE), fetal bovine serum, bovine brain extract, hydrocortisone, insulin, phenol red, phorbol myristate acetate (PMA), epidermal growth factor (EGF), nerve growth factor, transferrin, epinephrine, calcium chloride, penicillin, streptomycin, gentamycin, Amphotericin B, cholera toxin, triiodothyronine, and combinations thereof. Proliferation factors can be added to the culture medium at concentrations ranging between about 1 fg/ml to 1 mg/ml. Concentrations between about 1 to 100 ng/ml are usually sufficient. Simple titration experiments can be easily performed to determine the optimal concentration of a particular proliferation factor. An example of a proliferation factor contains about 2% fetal bovine serum, about 10 ng/ml epidermal growth factor, about 10⁻⁹ M triiodothyronine, about 5×10⁻⁵ M hydrocortisone, about 10 μg/ml insulin, about 10 μg/ml transferring, about 100 ng/ml 7S nerve growth factor, about 10⁻¹⁰ M cholera toxin, and about 150 μg/ml bovine brain extract.

The compositions can optionally comprise a pigment. The pigment can be a tattoo ink. The tattoo ink can be a permanent or non-permanent tattoo ink. The pigment can be selected based on the subject's natural skin color.

The cells capable of producing melanin and compositions described herein can also be used for purposes such as transplantation of cells or skin grafts containing transplanted cells into the skin. One can administer the cells to individuals desiring treatment for pigmentation loss in a manner similar to that used for conventional hair transplants. Cells capable of producing melanin are particularly useful for treating or preventing pigmentation loss, such as caused by vitiligo. As such, pigmentation loss is combated by the ability of the cells to produce melanin. For example, transplantation of melanin stem cells into the skin can increase the number of cells capable of producing melanin in individuals suffering from conditions characterized by hypopigmentation or loss of pigmentation such as vitiligo.

The method of treatment involves the preparation of the recipient site with a laser that creates microscopic recipient micropore channels or voids. The treatment parameters for the laser can be selected so as to produce micropore channels or voids of the desired width and depth, so as to produce the desired density of micropore channels or voids. The treatment parameters for the laser can be selected so as to produce particular characteristics in the micropore channels, such as the extent of the thermal coagulation zone. The treatment parameters for the laser can be selected so as to produce particular characteristics in the voids, such as the extent of the thermal ablation zone, char zone, and/or thermal coagulation zone.

The diameter of the micropore channels or voids created by the laser can be between about 5 μm and about 250 μm, between about 10 μm and about 110 μm, or between about 10 μm and about 25 μm. The micropore channels or voids can extend as deep as into the basal layer of the epidermis, into the dermal-epidermal junction of the skin, or into the dermis of the skin. The depth of the micropore channels or voids can be between about 30 μm and about 300 μm, between about 40 μm and about 200 μm, or between about 50 μm and about 150 μm. The diameter and depth of the micropore channels or voids can be selected based on the part of the body to be treated.

The diameter of the micropore channels or voids created by the laser can be selected so as to allow for the placement of only one cell capable of producing melanin per micropore channel or void, or so as to allow for the placement of more than one cell capable of producing melanin per micropore channel or void. The micropore channel or void diameter can also be selected so as to control the level and pattern of cells capable of producing melanin and/or pigment deposited into the skin. The micropore channel or void density and the cell density used in the treatment can be used to control the level of pigmentation deposited into the skin. For example, if a subject's normally pigmented skin contains a melanocyte to keratinocyte ratio of approximately 1:10, depositing melanocytes to the depigmented region using a density of 7 μm of every 105 μm could provide a level of pigmentation in a depigmented region that would appear similar to the unaffected regions of the subject's skin. Alternatively, if a subject's normally pigmented skin to contains a melanocyte to keratinocyte ratio of approximately 1:20, depositing melanocytes to the hypopigmented region using a density of 7 μm of every 235 μm could provide a level of pigmentation in a depigmented region that would appear similar to the unaffected regions of the subject's skin.

Overall, the micropore channels or voids should be of a diameter, depth and density such that the micropore channels or voids will heal in a reasonably short period of time, such as, for example, between about 1 hour and about 48 hours. During the healing process, the epidermal layers of the skin should heal the micropore channels or voids, sealing the cells capable of producing melanin within the micropore channels or voids. Invagination from the surrounding epidermal stem cells could help this process. Using laser treatment parameters which produce a thermal coagulation zone and/or a minimal char zone in the voids produces voids more likely to sustain the cells capable of producing melanin, and reduces the likelihood that the cells will invaginate into the dermis rather than the epidermis upon healing.

The recipient site can be prepared by introduction of a commercially available scaffolding material such as but not limited to poly(lactic-co-glycolic acid) (PLGA), fibronectin, collagen 1, or collagen 3. The scaffolding material can include a self-assembling molecule, such as, for example, a peptide hydrogel, a carbon nanotube, and mixtures thereof. The scaffolding material can be introduced after laser injury but prior to cell transplantation. Cells can be transplanted by applying a stem cell impregnated biogel dressing onto the laser injured recipient site. The recipient site covered by the cellular dressing would then be sealed using petrolatum and tegaderm or another biological dressing.

Implanted stem cells (e.g., constituted by hair bulge stem cells, melanoblasts and/or melanocyte stem cells used in isolation or in combinations ranging from about 0 to about 100% each) can be differentiated in vivo by steps using serum free media supplemented with inductive growth factors.

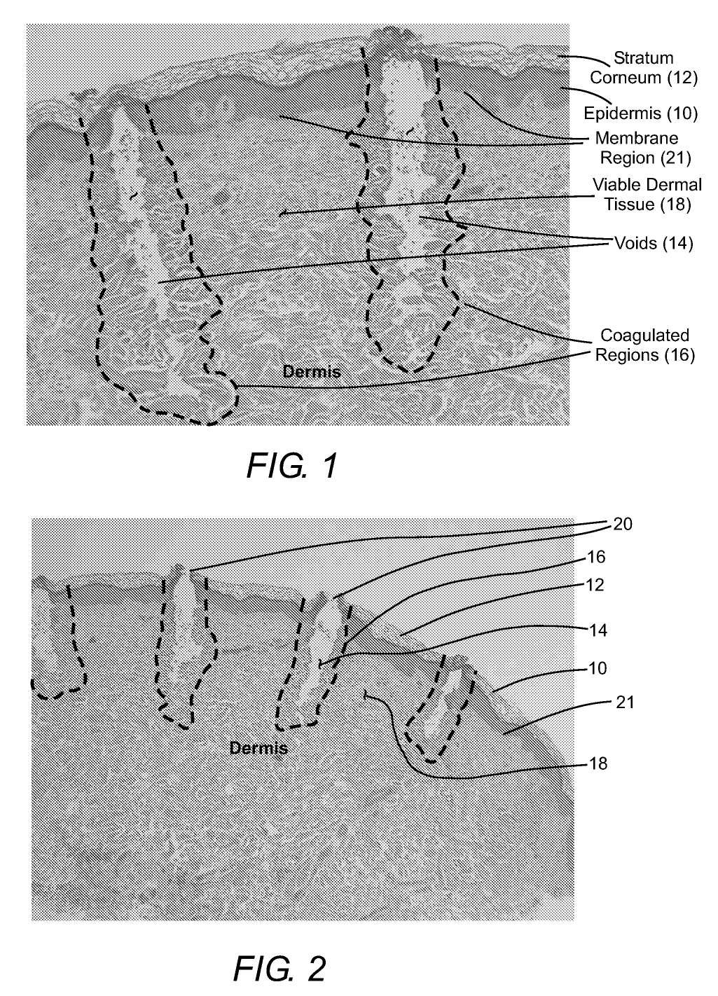

Referring now to the drawings, wherein like features are designated by like reference numerals, FIG. 1 and FIG. 2 are micrographs schematically illustrating a section of human skin immediately after irradiation with laser radiation to provide voids capable of receiving cells in accordance with the method of the present invention. FIG. 2 is at twice the magnification of FIG. 1. The skin was irradiated at spaced-apart locations with pulses of radiation having a wavelength of 10.6 micrometers (μm) from a CO₂ laser delivering a substantially TEM₀₀-quality beam. Each location was irradiated by one pulse. The radiation at the locations was focused to a spot having a diameter of about 120 μm at the surface of the skin, expanding slightly to between about 150 μm and 170 μm at a depth of about 1 mm in the skin. The laser output was repetitively pulsed at a pulse repetition frequency (PRF) of about 60-100 Hz. The pulses were nominally “square” laser pulses having a peak power of about 40 Watts (W) and a pulse duration of about 0.5 milliseconds (ms) to produce a pulse energy of 20 millijoules (mJ). The pulse duration could be varied to create different pulse energies for other experimental treatments. Experimental evaluations were performed with pulse energies in a range between about 5 mJ and 40 mJ. Laser pulses were scanned over the surface using a scanner wheel device to provide the spaced apart voids. The PRF of the laser was synchronized with the rotation of the scanner wheel. A detailed description of a preferred example of such a scanner wheel is presented further herein below.

The skin tissue includes a bulk dermal portion or dermis covered by an epidermal layer (epidermis) 12 typically having a thickness between about 30 μm and 150 μm. The top layer of the epidermis is covered, in turn, by a stratum corneum layer 10 typically having a thickness between about 5 μm and 15 μm. Tissue was ablated at each pulse location, producing a plurality of spaced-apart voids 14, elongated in the direction of incident radiation, and extending through the stratum corneum and the epidermis into the dermis.

In the example of FIGS. 1 and 2, the voids with the parameters mentioned above have an average diameter (width) of between about 180 μm and 240 μm. These dimensions are provided merely for guidance, as it will be evident from the micrographs that the diameter of any one void varies as the result of several factors including, for example, the inhomogeneous structure and absorption properties of the tissue. The voids have an average depth of between about 800 μm and 1000 μm, and are distributed with a density of approximately 400 voids per square centimeter (cm²). Walls of the voids are substantially cauterized by heat generated due to the ablation, thereby minimizing bleeding in and from the voids. This heat also produces a region 16 of coagulated tissue (coagulum) surrounding each void. Note that the term “surrounding” as used in this application does not imply that there is tissue remaining above the void. Here, the void is defined as being surrounded by coagulated tissue if dermal tissue around the walls of the void is coagulated. The void is defined as the region that is ablated. Immediately following ablation the voids are open. The appearance of closure of some voids in FIG. 2 is believed to be an artifact of the preparation of tissue samples for microscopic evaluation.

The coagulated regions have a thickness between about 20 μm and 80 μm immediately after ablation of the voids. Here again, however, thickness varies randomly with depth of the void because of above-mentioned factors affecting the diameter of the void. Between each void 14 and the surrounding coagulum 16 is a region of 18 of viable tissue. This includes a viable region of the stratum corneum, the epidermis, and the dermis. The region of viable tissue can have a width, at a narrowest point thereof, at least about equal to the maximum thickness of the coagulated regions 16 to allow sufficient space for the passage of nutrients to cause rapid healing and to preserve an adequate supply of transit amplifying cells to perform the reepithelialization of the wounded area. The viable tissue separating the coagulated tissue around the voids has a width, at a narrowest point thereof, between about 50 μm and 500 μm. The density of micropore channels or voids can be between about 200 and about 4000 micropore channels or voids per cm². The density of micropore channels or voids can be between about 1000 and about 2000 per cm². The density of treatment zones can be higher than the desired hair density because not every cell implantation sites will result in the production of melanin. This micropore channel or void density can be achieved in a single pass or multiple passes of a treatment device of applicator, for example two to ten passes, in order to minimize gaps and patterning that may be present if micropore channels or voids are created in a single pass of the applicator.

Heat from the ablation process that causes the coagulation in regions 16 effectively raises the temperature of the collagen in those coagulated regions sufficiently to create dramatic shrinkage or shortening of collagen in the coagulated tissue. This provides a hoop of contractile tissue around the void at each level of depth of the void. Upon collagen shrinkage, the dermal tissue is pulled inward, effectively tightening the dermal tissue. This tightening pulls taut any overlying laxity through a stretching of the epidermis and stratum corneum. This latter response is primarily due to the connection of a basement membrane region 21 of the epidermis to the collagen and elastin extra-cellular matrix. This connection provides a link between the epidermis and dermis. The contractile tissue very quickly shrinks the void, and creates an increase in skin tension resulting in a prompt significant reduction in overall skin laxity and the appearance of wrinkles. This shrinkage mechanism is supplemented by a wound-healing process healing described below.

Closure of the micropore channel or void occurs within a period of about 48 hours or less through a combination of the wound healing response in the case of micropore channels, and the above-described prompt collagen shrinkage and the subsequent wound healing response in the case of voids. The wound healing process begins with re-epithelialization of the perimeter of the void, which typically takes less than 24 hours, formation of a fluid filled vacuole, followed by infiltration by macrophages and subsequent dermal remodeling by the collagen and elastin forming fibroblasts. The column of coagulated tissue has excellent mechanical integrity that supports a progressive remodeling process without significant loss of the original shrinkage. In addition, the coagulated tissue acts as a tightened tissue scaffold with increased resistance to stretching. This further facilitates wound healing and skin tightening. The tightened scaffold serves as the structure upon which new collagen is deposited during wound healing and helps to create a significantly tighter and longer lasting result than would be created without the removal of tissue and the shrinkage due to collagen coagulation.

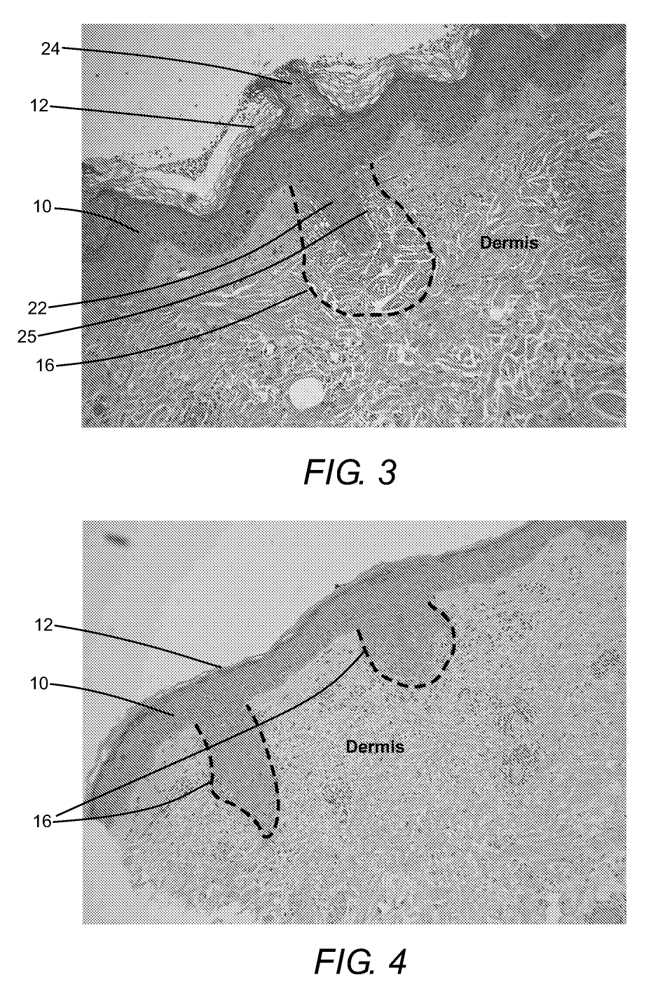

Progress of the healing after a period of about 48 hours from the irradiation conditions of FIG. 1 is illustrated by the micrograph of FIG. 3, which has the same magnification. Here, the coagulated region 16 is reduced both in diameter and depth compared with a comparable region of FIG. 1. In the micrograph of FIG. 3 epidermal stem cells have migrated into the void and facilitated healing of the void area. Epidermal stem cells proliferate and differentiate into epidermal keratinocytes filling the void in a centripetal fashion. As epidermal cells proliferate and fill the void, the coagulated material is pushed up the epidermis toward the stratum corneum. The voids contain microscopic-epidermal necrotic debris (MEND). The pushing of the coagulated material forces a plug 24 of the MEND to seal the stratum corneum during the healing response, thus preventing access of the outside environment to the inside of the skin.

At this time, the basement membrane is ill-defined and has yet to be completely repaired and restored. This is clearly depicted by the vacuolar space 25 separating the healed void and the dermis. In FIGS. 1 and 2, there is sparse cellularity evident in the dermis. However, in the micrograph of FIG. 3, the wound healing response at 48 hours has led to increased release of signaling molecules, such as chemokines, from the area of spared tissue, leading to recruitment of inflammatory cells aiding in the healing response.

Progress of the healing after a period of about one week from the irradiation conditions of FIG. 1 is illustrated by the micrograph of FIG. 4. Here, the MEND has been exfoliated. The void has been replaced by epidermal cells which gradually remodel to create a normal rete ridge pattern, reducing in depth of invagination. The healing process has triggered that some of the deeper epidermal cells go through apoptosis, thereby disappearing from the replaced void tissue. The basement membrane of the epidermis has almost fully been restored as evidenced by the lack of vacuolization between the epidermis and dermis. During the wound healing response, cytokines such as TGF beta, amongst others, are released and allow fibroblasts to secrete collagen, elastin, and extracellular matrix. This secreted matrix replaces the apoptotic epidermal cells of the void. The coagulated dermal tissue has been replaced by a similar process sparked by the laser irradiation treatment induced release of pro-neocollagenesis cytokines. Inflammatory cells also help remove non-viable debris in the dermis, allowing the replacement of coagulated tissue with fresh viable tissue as outlined above.

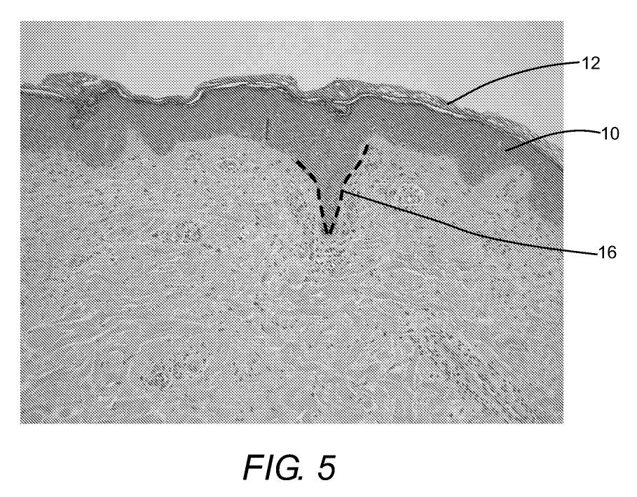

FIG. 5 depicts progress of healing one month after initial treatment. Here remodeling of the void has continued by apoptosis of the deeper epidermal cells, leading to a more natural rete ridge like structure. The MEND is absent, and the basement membrane of the epidermis is completely healed. Inflammatory cells are still present in the dermis, and fibroblasts continue to lay down new matrix in the dermis. This provides that over the ensuing two to six months, new collagen synthesis continues to replace previously coagulated dermal tissue, providing for increased tensile strength in the dermis.

The complete replacement of the coagulated tissue providing the initial skin tightening with new collagen and elastin as described above provides for a long lasting improvement in the appearance of wrinkles in temporally or photo aged skin. As the inventive method results in a completely healthy treated area once the healing process is complete, an area of skin treated once can be treated again, for example, after a period of about two months to provide further improvement or adjustment of skin color. Clearly, however, the progress of skin aging and loosening can not be arrested permanently, and the length of time that any improved appearance will be evident will depend on the age of the person receiving the treatment and the environment to which treated skin is exposed, among other factors. Also, due to the autoimmune nature of some cases of vitiligo, it may be necessary to repeatedly implant new cells capable of producing melanin to maintain a ‘normal’ level of pigmentation in the skin.

In the example described above, skin irradiation for void formation was performed with laser radiation having a wavelength (10.6 μs) that is strongly absorbed by water. The radiation was delivered as a beam having TEM₀₀ quality, or near TEM₀₀ quality. The CO₂ laser used in the example of the present invention discussed above is a relatively simple and relatively inexpensive laser for providing such a beam. The 10.6 μm radiation of a CO₂ laser has an absorption coefficient in water of approximately 850 inverse centimeters (cm⁻¹).

To efficiently ablate tissue, a high absorption coefficient in the water of the skin tissue is desired. However, in order to form a coagulation region surrounding the voids, to cause tissue shrinkage and to reduce bleeding at the treatment sites, the absorption coefficient should not be too high. Alternatively, to produce micropore channels, the absorption coefficient should not be too high. The laser radiation used in the inventive method to produce micropore channels or voids can have an absorption coefficient in water in the range between about 50 cm⁻¹ and about 12,300 cm⁻¹. The absorption coefficient can be between about 75 cm⁻¹and about 850 cm⁻¹, between about 2,000 cm⁻¹ and about 6,000 cm⁻¹, or between about 11,500 cm⁻¹ and about 12,500 cm⁻¹. The absorption coefficient can be about 82 cm⁻¹, about 780 cm⁻¹, about 820 cm⁻¹, about 2,700 cm⁻¹, about 5,000 cm⁻¹, or about 12,200 cm⁻¹.

At each of the above absorption levels, the laser pulse for forming the micropore channels or voids can have a duration between about 100 microseconds (μs) and about 5 ms. The actual treatment parameters can be chosen based on commercial tradeoffs of available laser powers and desired treatment-zone sizes.

Lasers providing radiation having a wavelength that has an absorption coefficient in water in the above ranges include CO₂, CO, and free-electron lasers (500-1000 cm⁻¹), thulium-doped fiber lasers and free-electron lasers (50-1000 cm⁻¹), Er:YAG lasers, erbium, chromium: yttrium-scandium-gallium-garnet (Er,Cr:YSGG) lasers, Raman-shifted erbium-doped fiber lasers, and free-electron lasers (between about 100 cm⁻¹ and 12,300 cm⁻¹). Tunable lasers, such as, for example, chromium: zinc-selenium (Cr:ZnSe) tunable 2.4-3.0 μm lasers, can be used as well. Other light sources, such as optical parametric oscillators (OPOs), including 6.1 μm or 6.45 μm OPOs, and laser pumped optical parametric amplifiers (OPAs) can also be used.

The micropore channels or voids 14 can have a diameter between about 10 μm and about 500 μm, between about 50 μm and about 400 μm, or between about 100 μm and about 300 μm. The micropore channels or voids can have a diameter between about 40 μm and about 150 μm. The micropore channels or voids can be spaced apart with a center to center distance of between about 200 μm and 1500 μm depending on the size of the micropore channels or voids 14 and the coagulated regions 16. The center to center distance can be chosen based on the level of desired treatment. A coverage area for the coagulated regions and micropore channels or coagulated regions and voids immediately following treatment is preferably between about 5% and 50% of the treated area. A higher level of coverage will be more likely to have a higher level of side effects for a similar treatment energy per treatment site.

The depth of the micropore channels or voids can be within the basal level of the epidermis, below the basal level of the epidermis within the dermal-epidermal junction of the skin, or below the dermal-epidermal junction of the skin in the papillary dermis. Depending upon the region of the body and the thickness of the skin, these regions can range between about 30 μm and about 500 μm in depth. The micropore channels or voids can be randomly distributed over an area of skin being treated.

In relative and practical terms, the micropore channels or voids can be placed such that coagulated zones 16 surrounding the micropore channels or voids are separated by at least the average thickness of the coagulated zones. This can be determined by making micrographs of test irradiations, similar to the above-discussed micrographs of FIGS. 1 and 2. If the micropore channels or voids are too closely spaced, the healing process may be protracted or incomplete. If the micropore channels or voids are spaced too far apart, more than one treatment may be necessary to achieve an acceptable level of pigmentation. For purposes of implanting cells capable of producing melanin into the skin, the depth of the micropore channels or voids can extend into the basal level of the epidermis, below the basal level of the epidermis and into the dermal-epidermal junction of the skin, or below the dermal-epidermal junction of the skin and into the papillary dermis.

In order to produce the micropore channels or voids as described above, a range of possible radiant exposures can be used, depending upon the type of laser used. Radiant exposures of between about 0.05 J/cm² and about 90 J/cm² can be used. Radian exposures of between about 0.05 J/cm² and about 10 J/cm², or between about 11 J/cm² and about 90 J/cm² can be used. Similarly, the pulse energy used can range between about 2 μJ and about 550 μJ. The pulse energy used can range between about 1 μJ and about 10 μJ, between about 12 and about 20 μJ, or between about 100 μJ and about 550 μJ.

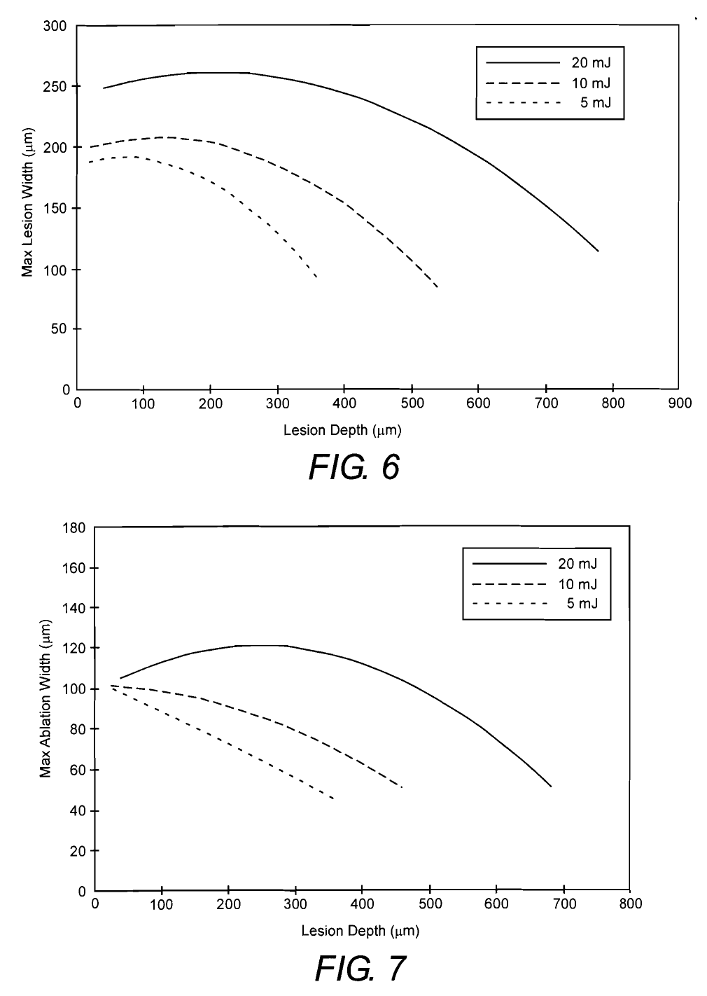

FIG. 6 and FIG. 7 are graphs schematically illustrating respectively trends for maximum width of the a treatment zone (lesion), i.e., maximum total width of a void 14 plus surrounding coagulated region 16, and maximum width of the void (ablated region), as a function of lesion depth, i.e., the depth to the base of the coagulated region. The trends in each graph are shown for pulse energies of 5 mJ, 10 mJ, and 20 mJ. It should be noted here that these trends fitted through a number of experimental measurements with relatively wide error bars, particularly at shallow lesion depth. Accordingly, it is recommended that these graphs be treated as guidelines only.

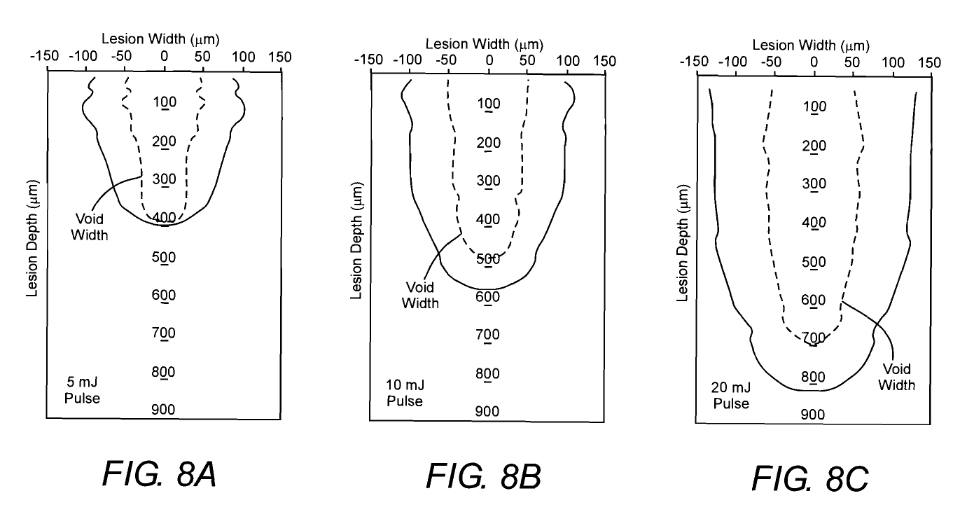

FIG. 8A, FIG. 8B, and FIG. 8C are graphs schematically illustrating graphical lesion width (solid curves) and void width (dashed curves) as a function of lesion depth for experimental irradiations at respectively 5 mJ, 10 mJ, and 20 mJ. These graphs are derived from measurements taken from micrographs of transverse sections through the experimental legions. The graphs of FIGS. 7 and 8A-C can be used as guidelines to select initial spacing of treatment zones in the inventive method. This spacing can then be optimized by experiment.

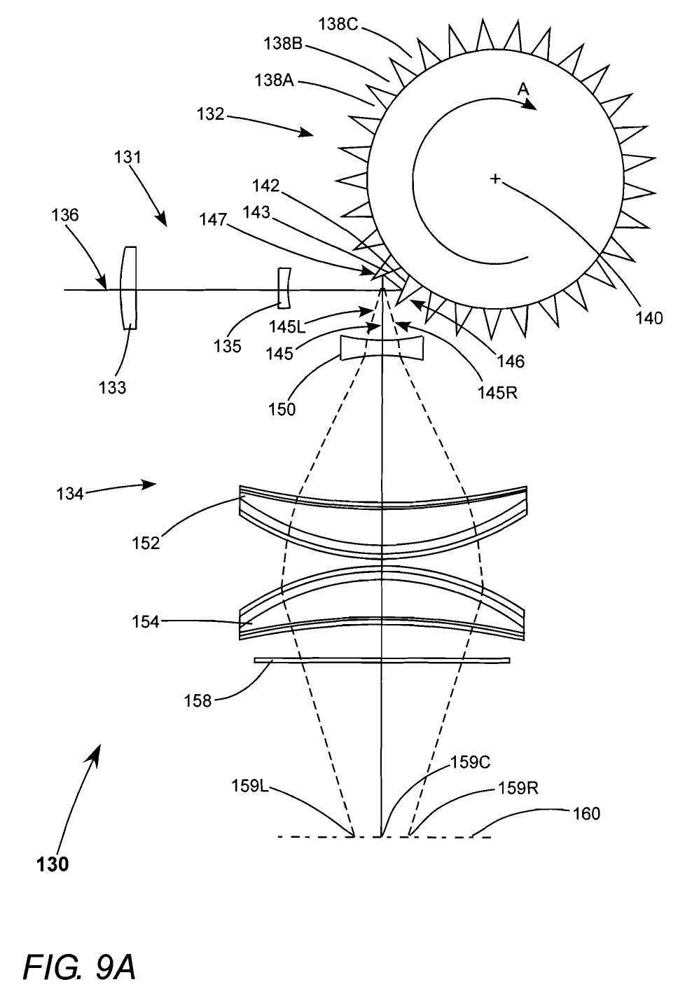

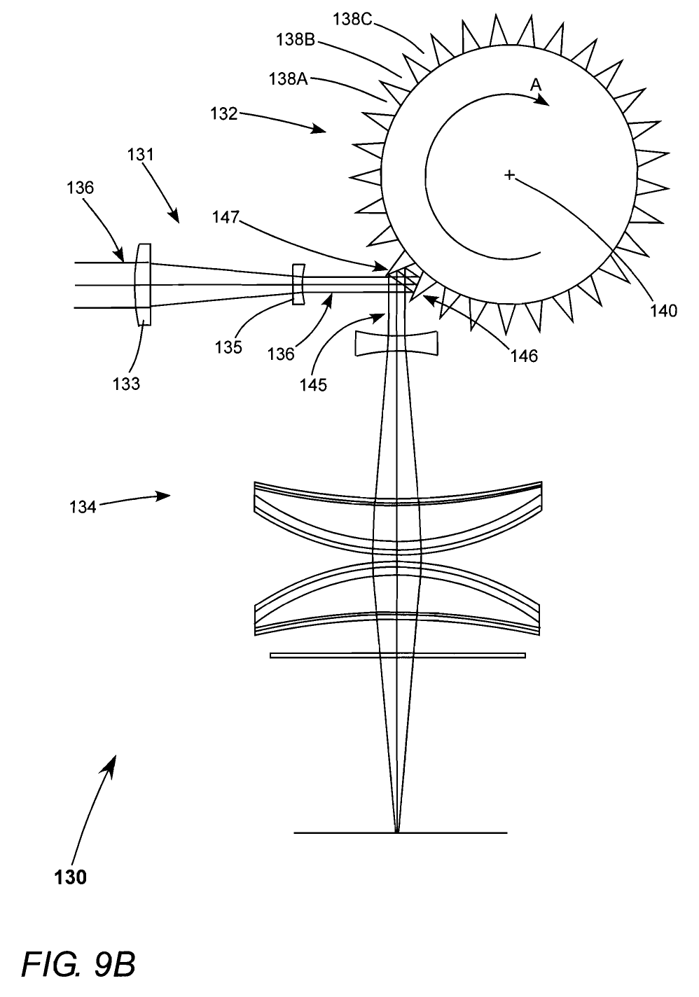

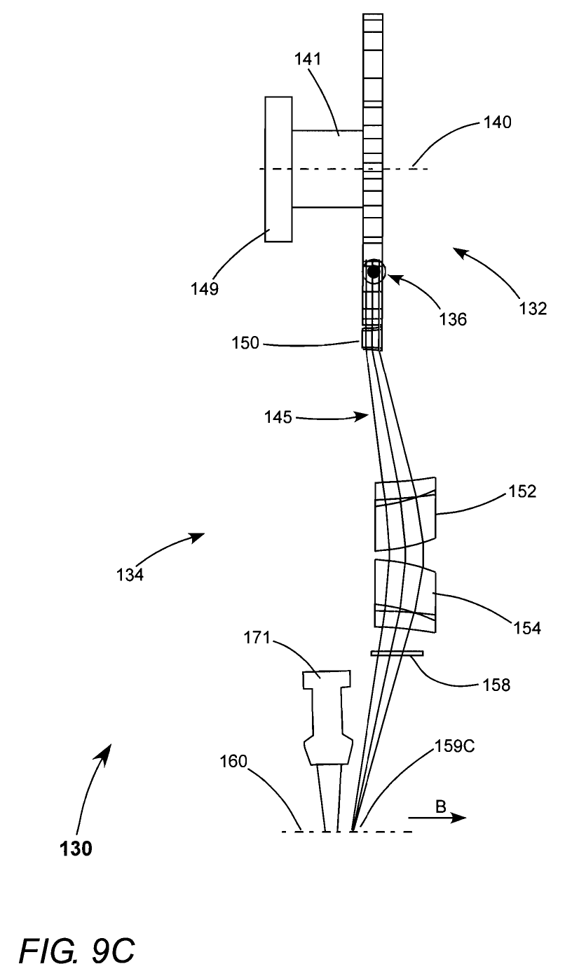

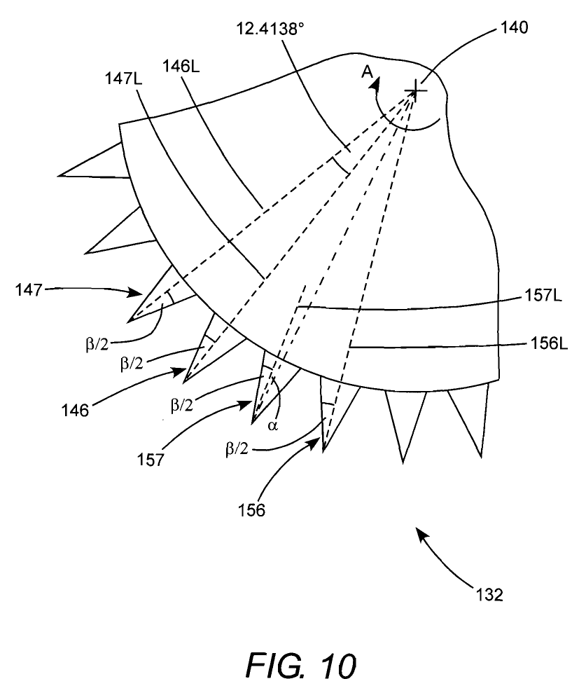

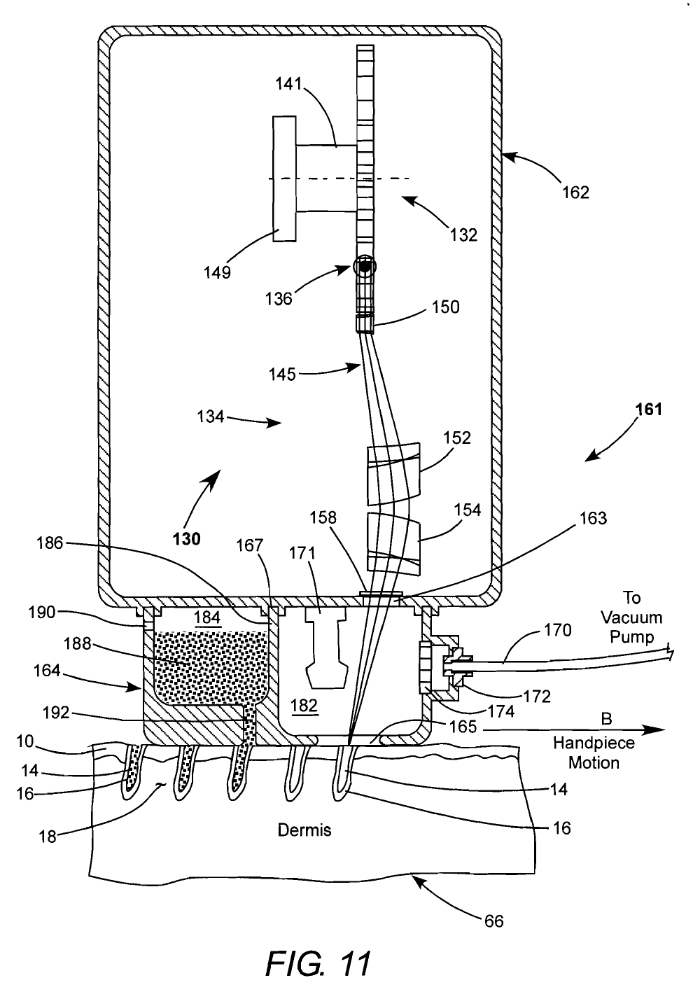

In any area being treated, all micropore channels or voids can be created simultaneously. However, apparatus capable of simultaneously creating an effective number of micropore channels or voids with appropriate spacing over a useful area of skin may not be practical or cost effective. Practically, the micropore channels or voids can be created sequentially, but because of the rapid onset of the healing process, it is preferable that sequential treatment of tissue to create the micropore channels or voids in an area being treated is completed in a time period less than about 60 minutes (min). It is possible to create micropore channels or voids at a rate between about 10 Hz and 5000 Hz and or at a rate between about 100 Hz and 5000 Hz, because these rates reduce the physician time for treatment. Increasing the treatment rate above 5000 Hz causes the laser and scanning systems to be more expensive and therefore less commercially desirable, even though they are technologically feasible using the apparatus presented here. One example of an apparatus in accordance with the present invention for providing rapid sequential delivery of optical pulses and immediately thereafter introducing cells and growth factor into the micropore channels or voids is described below with reference to FIG. 9A, FIG. 9B, FIG. 9C, FIG. 10, and FIG. 11. FIGS. 9A-C and FIG. 10 depict apparatus for ablating the voids and FIG. 10 depicts an applicator including the void-ablating apparatus and means for introducing the cells and growth media into the voids.

Beginning with a description of the laser apparatus, FIG. 9A is a front elevation view schematically illustrating an ablation apparatus 130 including a scanner wheel 132 and a wide field projection lens 134. The scanner wheel is driven by a motor 149 via a hub 141 (see FIG. 9C). Scanner wheel 132 is arranged to receive an incident laser beam 136 lying substantially in the plane of rotation of the scanner wheel. In FIG. 9A beam 36 is represented by only a single principle ray. FIG. 9B and FIG. 9C are respectively front and side elevation views of apparatus in which beam 36 is represented by a plurality of rays.

Before being incident on the scanning wheel, beam 136 is compressed (see FIG. 9B) by a telescope 131 comprising a positive lens 133 and a negative lens 135. In this example, the scanner wheel is divided into twenty nine sectors 138A, 138B, 138C, etc., which are arranged in a circle centered on the rotation axis 140 of the scanner wheel. The wheel, here, is assumed to rotate in a clockwise direction as indicated by arrow A. The incident laser beam 136 propagates along a direction that lies in the plane of rotation. Each sector 138 of scanner wheel 132 includes a pair of reflective elements, for example, reflective surfaces 142 and 143 for the sector that is indicated as being active. The surface normals of the reflective surfaces have a substantial component in the plane of rotation of the scanner wheel. In this example, the scanner wheel includes prisms 146, 147, etc. that are arranged in a circle. The faces of the prisms are reflectively coated and the reflectively coated surfaces of adjacent prisms, for example, reflective surfaces 142 and 143 from prisms 146 and 147, form the opposing reflective surfaces for a sector. Alternatively, the reflective surfaces can be metal surfaces that are polished to be smooth enough to cause sufficient reflectivity.

Each sector 138 deflects the incoming optical beam 136 by some angular amount. The sectors 138 are designed so that the angular deflection is approximately constant as each sector rotates through the incident optical beam 136, but the angular deflection varies from sector to sector. In more detail, the incident optical beam 136 reflects from the first reflective surface 132 on prism 146, and subsequently reflects from reflective surface 143 on prism 147 before exiting as output optical beam 145.

The two reflective surfaces 142 and 143 form a Penta mirror geometry. An even number of reflective surfaces that rotate together in the plane of the folded optical path has the property that the angular deflection of output beam 145 from input beam 136 is invariant with the rotation angle of the reflective surfaces. In this case, there are two reflective surfaces 142 and 143 and rotation of the scanner wheel 132 causes the prisms 146 and 147 and reflective surfaces 142 and 143 thereof to rotate together in the plane of the folded optical path. As a result, the output beam direction does not change as the two reflective surfaces 142 and 143 rotate through the incident optical beam 136. The beam can be focused at the treatment surface such that the beam does not walk across the surface during the scanning or the beam can be used at another plane such that the beam walks across the surface during the scanning due to the translation of the beam in a conjugate plane that translates into an angular variation during the scanning due to the rotation of the scanning wheel. The reflective surfaces 142 and 143 are self-compensating with respect to rotation of scanner wheel 32. Furthermore, as the reflective surfaces 142 and 143 are planar, they will also be substantially spatially invariant with respect to wobble of the scanner wheel.

As the scanner wheel rotates clockwise to the next sector 138 and the next two reflective surfaces, the angular deflection can be changed by using a different included angle between the opposing reflective surfaces. For this configuration, the beam will be deflected by an angle that is twice that of the included angle. By way of example, if the included angle for sector 138A is 45 degrees, sector 138A will deflect the incident laser beam by 90 degrees. If the included angle for sector 138B is 44.5 degrees, then the incident laser beam will be deflected by 89 degrees, and so on. In this example, different included angles are used for each of the sectors so that each sector will produce an output optical beam that is deflected by a different amount. However, the deflection angle will be substantially invariant within each sector due to the even number of reflective surfaces rotating together through the incident beam. For this example, the angular deflections have a nominal magnitude of 90 degrees and a variance of −15 to +15 degrees from the nominal magnitude. Beam 145 in extreme left and right scanning positions is indicated by dashed lines 45L and 45R respectively. Here again, in FIG. 9A beam 145 is represented by only a single principle ray, while FIG. 9B and FIG. 9B represent beam 145 by a plurality of rays.

Referring in particular to FIG. 10, in this example of scanner wheel 132, the apex angle of each prism is 32.5862 degrees, calculated as follows. Each sector 138 subtends an equal angular amount. Since there are twenty nine sectors, each sector subtends 360/29=12.4138 degrees. The two prisms 146 and 147 have the same shape and, therefore, the same apex angle β. Scanner wheel 132 is designed so that when the included angle is 45 degrees, the prisms 146 and 147 are positioned so that lines 147L and 146L that bisect the apex angle of prisms 146 and 147 also passes through the rotation axis 140. Accordingly, the design must satisfy an equation β/2+12.4138+β/2=45. Solving this equation yields an apex angle of β=32.5862 degrees.

The next prism 157 moving counterclockwise on scanner wheel 132 from prism 146 is tilted slightly by an angle +α so its bisecting line 157L does not pass through the center of rotation 140 of the scanner wheel. As a result, the included angle for the sector formed by prisms 146 and 157 is (β/2+α)+12.4138+β/2=45+α. The next prism 156 is once again aligned with the rotation center 140 (as indicated by bisecting line 56L), so the included angle for the sector formed by prisms 56 and 57 is (β/2−α)+12.4138+β/2=45−α. The next prism is tilted by +2 α, followed by an aligned prism, and then a prism tilted by +3 α, followed by another aligned prism, etc. This geometry is maintained around the periphery of the scanner wheel. This specific arrangement produces twenty nine deflection angles that vary over the range of −15 degrees to +15 degrees relative to the nominal 90 degree magnitude. Note that this approach uses an odd number of sectors where every other (approximately) prism is aligned and the alternate prisms are tilted by angles α, 2α, 3α, etc. In an alternate embodiment, the surface on which beam 136 is incident has zero tilt and all tilt is taken up in the reflective surface on the second facet.

Wide field lens 134, here includes optical elements 150, 152, and 154, and an output window 158. In the lens depicted in FIGS. 9A-C the optical elements are assumed to made from zinc selenide which has excellent transparency for 10.6-micrometer radiation. Those skilled in the art will recognize that other IR transparent materials such as zinc sulfide (ZnS) or germanium (Ge) may be used for elements in such a lens with appropriate reconfiguration of the elements. Optical elements 152, 154, and 156 are tilted off axis spherical elements. Lens 134 focuses exit beam 145 from scanner wheel 132 in a plane 160 in which skin to be treated would be located. Lens 134 focuses exit beam 145 at each angular position that the beam leaves scanner wheel 132. This provides a line or row sequence of 29 focal spots (one for each scanning sector of the scanner wheel) in plane 160. In FIG. 9A three of those spots are designated including an extreme left spot 159L, a center spot 159C and an extreme right spot 159R. The remaining 26 spots (not shown) are approximately evenly distributed between spots 159L, 159C, and 159R. Another line of focal spots can be produced by moving apparatus 130 perpendicular to the original line as indicated in FIG. 9C by arrow B.

Referring in particular to FIG. 9C, the tilted off-axis spherical elements 150, 152 and 154 are arranged such that beam 145 is first directed, (by bi-concave negative) lens element 150, away from the plane of rotation of the scanner wheel. Elements 152 and 154 (positive meniscus elements) then direct the beam back towards the plane or rotation, while focusing the beam, such that the focused beam is incident non-normally (non-orthogonally) in plane 160, i.e., normal to skin being treated. One particular of this non-normal incidence of beam 145 on the skin is that window 158 and optical element 154 are laterally displaced from the focal point and are removed from the principal path of debris that may be ejected from a site being irradiated. Another advantage is that a motion senor optics for controlling firing of the laser in accordance with distance traveled by the apparatus, for example, an optical mouse or the like, designated in FIG. 9C by the reference numeral 171, may be directed close to the point of irradiation. This is advantageous for control accuracy. As far as the actual treatment is concerned, it is not believed that there is any advantage of non-orthogonal compared with non-orthogonal (normal incidence) irradiation.

Those skilled in the art will recognize that it is not necessary that all sectors of the scanner wheel have a different deflection angle. Prisms of the scanning wheel can be configured such that groups of two or more sectors provide the same deflection angle with the deflection angle being varied from group to group. Such a configuration can be used to provide less voids in a row with increased spacing there between. It is also not necessary that deflection angle be increased or decreased progressively from sector to sector. It is preferred in that pulsed operation of the laser providing beam 136, that the PRF of the laser is synchronized with rotation of the scanner wheel such that sequential sectors of the wheel enter the path of beam 136 to intercept sequential pulses from the laser.

It should be noted here that apparatus 130 including scanner wheel 132 and focusing lens 134 is one of several combinations of scanning and focusing devices that could be used for carrying out the method of the present invention and the description of this particular apparatus should not be construed as limiting the invention. By way of example, different rotary scanning devices and focusing lenses are described in U.S. patent application Ser. No. 11/158907, filed Jun. 20, 2005, the complete disclosure of which is hereby incorporated by reference. Galvanometer-based reflective scanning systems can also be used to practice this invention and have the advantage of being robust and well-proven technology for laser delivery. Scanning rates with a galvanometer-based reflective scanning systems, however, will be more limited than with the a scanner such as scanning wheel 132 described above, due to the inertia of the reflective component and the changes of direction required to form a scanning pattern over a substantial treatment area. Other scanner systems can be used and are well known in the art.

FIG. 11 schematically illustrates one embodiment of a handpiece 161 or applicator in accordance with the present invention including an example of above described apparatus 130. Handpiece 161 is depicted irradiating a fragment 166 of skin being treated. The handpiece is moved over the skin being treated, as indicated by arrow B, with tip 164 in contact with the skin. The irradiation provides parallel spaced-apart rows of above-described spaced-apart voids 14, only end ones of which are visible in FIG. 8. Spacing between the rows of spots may be narrower or broader than that depicted in FIG. 8, the spacing, here, being selected for convenience of illustration. Control of the row spacing can be affected by controlling delivery of the laser beam by optical motion sensor 171, or alternatively a mechanical motion sensor (mechanical mouse), as is known in the art. A description of such motion sensing and control is not necessary for understanding principles of the present invention and accordingly is not presented here. Descriptions of techniques for controlling delivery of a pattern of laser spots are provided in U.S. patent application Ser. No. 10/888,356 entitled “Method and Apparatus for fractional photo therapy of skin” and Ser. No. 11/020,648 entitled “Method and apparatus for monitoring and controlling laser-induced tissue treatment,” the complete disclosures of which are hereby incorporated herein by reference.

In a preferred method of operation, apparatus 130 is housed in handpiece or applicator 161 including a housing 162 to which is attached an open-topped, removable tip 164, which is attached to the housing via slots 167. Pins and/or screws can also be used for this purpose. When tip 164 is attached to housing the tip is divided into two chambers 182 and 184 having no gas-passage there between. An aperture 163 in housing 162 is covered by window 158 such that optical access to chamber 182 is provided while preventing gas passage between the housing and chamber 182. In use, the base of the tip makes a reasonable gas-tight seal with the skin.

Laser beam 136 is directed into housing 162 via an articulated arm (not shown). Articulated arms for delivery infra red laser radiation are well known in the art. One preferred articulated arm is described in U.S. patent application Ser. No. 60/752,850 filed Dec. 21, 2005 entitled “Articulated arm for delivering a laser beam,” the complete disclosure of which is hereby incorporated herein by reference. The focused beam 145 from lens 134 exits housing 162 via exit window 158, (here attached to the housing) and via aperture 163 in the housing, then passes through chamber 182 of tip 164 exiting via aperture 165 therein. A vacuum pump (not shown) is connected to removable tip 164 via a hose or tube 170. Tube 170 is connected to tip 164 via a removable and replaceable adaptor 172. Operating the vacuum pump with tip 164 in contact with the skin creates negative pressure (partial vacuum) inside the tip. This withdraws smoke resulting from the laser ablation from the path of the laser beam, and draws debris products of the ablation away from window 158 in the housing. A filter element 174 in a wall of tip 164 prevents debris from being drawn into vacuum hose 170 and eventually into the pump.

The arrangement of the tip provides that, when the vacuum pump is operated, there is also negative pressure created in any void that is under the aperture. The seal of the base of the tip to the skin retains the negative pressure in voids over which the tip has passed. Chamber 184 of tip 164 serves as a reservoir for a mixture of cells and growth medium 188. A channel though the tip, from chamber 184 through the base of the tip, allows a flow of the stem-cell mixture into the voids. An aperture 190 through the tip allows gas to enter chamber 184 to assist in the free flow of the mixture through channel 192. It is also possible to supply positive pressure through such an aperture to further encourage flow of the mixture, where pressure is measured relative to the ambient pressure outside of the apparatus. Alternatively, the cells can be applied topically following laser irradiation without the assistance of vacuum or positive pressure.

### EXAMPLES

Having now generally described the invention, the same may be more readily understood through the following reference to the following examples. The examples are offered for illustrative purposes only, and are not intended to limit the scope of the present invention in any way. Efforts have been made to ensure accuracy with respect to numbers used (e.g., amounts, temperatures, etc.), but some experimental error and deviation should, of course, be allowed for.

### Example 1

Void creation

Freshly excised human skin samples are irradiated with a 30 W, 10.6 μm CO₂ laser at varying pulse energies. The laser beams carry a near diffraction limited 1/e² Gaussian spot size of approximately 120 μm, with pulse energies ranging from about 100 μJ to about 1000 μJ, providing radiant exposures between about 1 J/cm² and about 9 J/cm² that are delivered through an apparatus capable of a repetition rate up to 1500 spots/second.

The skin is heated on a digital hot plate (Cole-Parmer Instrument Co., Vernon Hills, Ill.), and the skin surface temperature is measured with a Mintemp MT4 infrared probe (Raytek Corporation, Santa Cruz, Calif.). The laser treatment is initiated when the skin surface reaches a temperature of 98±3° F. The laser handpiece is translated at a specific velocity by using a precision linear stage driven by an ESP 300 motion controller (Newport Co., Irvine, Calif.). The firing rate of the laser is automatically adjusted by the laser handpiece to produce a specific density of lesions. A single pass is made at a constant velocity of 1.0 cm/s and spot density of 400 microscopic ablative treatment zones per cm² creating an interlesional distance of approximately 500 μm. The voids thus created are about 30 μm to about 500 μm in depth. The treatment parameters are selected so as to produce voids as deep as the basal layer of the epidermis, the dermal-epidermal junction, or the papillary dermis.

### Example 2

Apparatus 2 for Micropore Channel or Void Creation

An apparatus comprising a 10.6 μm CO₂ laser capable of delivering varied pulse energies and a laser handpiece is assembled. The laser beams carry a near diffraction limited 1/e² Gaussian spot size of approximately 120 μm. The laser is capable of delivering pulse energies of at least between about 120 μJ and about 500 μJ. The apparatus is capable of providing radiant exposures between about 1 J/cm² and about 9 J/cm². The apparatus is capable of a repetition rate up to 1500 spots/second. The firing rate of the laser is automatically adjusted by the laser handpiece to produce a specific density of lesions. When used to treat skin, the apparatus produces micropore channels or voids between about 30 μm and about 500 μm in depth.

### Example 3

Apparatus 3 for Micropore Channel or Void Creation

An apparatus comprising a 1.925 μm thulium fiber laser capable of delivering varied pulse energies and a laser handpiece is assembled. The laser beams carry a near diffraction limited 1/e² Gaussian spot size of approximately 50 μm. The laser is capable of delivering pulse energies of at least about 200 μJ. The apparatus is capable of providing radiant exposures between about 11 J/cm² and about 85 J/cm². The apparatus is capable of a repetition rate up to 1500 spots/second. The firing rate of the laser is automatically adjusted by the laser handpiece to produce a specific density of lesions. When used to treat skin, the apparatus produces micropore channels or voids between about 30 μm and about 500 μm in depth.

### Example 4

Apparatus 4 for Micropore Channel or Void Creation

An apparatus comprising a 2.79 μm Er,Cr:YSGG laser capable of delivering varied pulse energies and a laser handpiece is assembled. The laser beams carry a near diffraction limited 1/e² Gaussian spot size of approximately 70 μm. The laser is capable of delivering pulse energies of at least about 5 μJ. The apparatus is capable of providing radiant exposures between about 0.17 J/cm² and about 1.5 J/cm². The apparatus is capable of a repetition rate up to 1500 spots/second. The firing rate of the laser is automatically adjusted by the laser handpiece to produce a specific density of lesions. When used to treat skin, the apparatus produces micropore channels or voids between about 30 μm and about 500 μm in depth.

### Example 5

Apparatus 5 for Micropore Channel or Void Creation

An apparatus comprising a 2.94 μm Er:YAG laser capable of delivering varied pulse energies and a laser handpiece is assembled. The laser beams carry a near diffraction limited 1/e² Gaussian spot size of approximately 75 μm. The laser is capable of delivering pulse energies of at least about 3 μJ. The apparatus is capable of providing radiant exposures between about 0.05 J/cm² and about 0.7 J/cm². The apparatus is capable of a repetition rate up to 1500 spots/second. The firing rate of the laser is automatically adjusted by the laser handpiece to produce a specific density of lesions. When used to treat skin, the apparatus produces micropore channels or voids between about 30 μm and about 500 μm in depth.

### Example 6

Apparatus 6 for Micropore Channel or Void Creation

An apparatus comprising a 6.1 μm OPO laser capable of delivering varied pulse energies and a laser handpiece is assembled. The laser beams carry a near diffraction limited 1/e² Gaussian spot size of approximately 40 μm. The laser is capable of delivering pulse energies of at least about 4 μJ. The apparatus is capable of providing radiant exposures between about 0.3 J/cm² and about 2.7 J/cm². The apparatus is capable of a repetition rate up to 1500 spots/second. The firing rate of the laser is automatically adjusted by the laser handpiece to produce a specific density of lesions. When used to treat skin, the apparatus produces micropore channels or voids between about 30 μm and about 500 μm in depth.

### Example 7

Apparatus 7 for Micropore Channel or Void Creation

An apparatus comprising a 6.45 μm OPO laser capable of delivering varied pulse energies and a laser handpiece is assembled. The laser beams carry a near diffraction limited 1/e² Gaussian spot size of approximately 40 μm. The laser is capable of delivering pulse energies of at least about 14 μJ. The apparatus is capable of providing radiant exposures between about 1 J/cm² and about 8.5 J/cm². The apparatus is capable of a repetition rate up to 1500 spots/second. The firing rate of the laser is automatically adjusted by the laser handpiece to produce a specific density of lesions. When used to treat skin, the apparatus produces micropore channels or voids between about 30 μm and about 500 μm in depth.

### Example 8

Treatment with Melanocytes

Voids are created in living skin using the apparatus described in Example 1. Into the base of the voids is implanted a solution containing melanocytes, a melanocyte growth medium, and PLGA that acts as a scaffolding material. Two to seven days following the treatment, the voids have healed, and the implanted melanocytes have become part of the basal layer of the epidermis. Following the treatment, the implanted melanocytes begin to produce melanin, which is subsequently taken up by the endogenous keratinocytes.

### Example 9

Micropore Channel Creation and Treatment with Melanocytes and Pigment

Micropore channels are created using a non-ablative laser apparatus. Laser treatment parameters are selected so as to produce micropore channels between about 50 μm and about 100 μm in diameter within the basal layer of the epidermis, between about 30 μm and about 300 μm in depth. Treatment parameters are also selected so as to produce the desired density of pigmentation following treatment. A solution containing melanocytes, a growth factor and a pigment is injected into the base of the micropore channels. Melanocyte growth is promoted by using serum free media containing a growth factor. The melanocytes attach to the skin the epidermis. Treatment with various growth media at various times following injection is used to help the melanocytes populate the micropore channels. During the healing process, the melanocytes grow but do not replicate. Over time, the combination of the pigment and the melanin produced by the melanocytes produces a level of pigmentation in the treated area that approximates the level of pigmentation of the subject's skin in areas unaffected by vitiligo. Repeated treatments are be provided to increase the level of pigmentation, or to replace melanocytes which do not implant. If the region of depigmentation expands over time, such regions can be treated as needed.

### Example 10

Micropore Channel or Void Creation and Treatment with Stem Cells

Micropore channels or voids are created in the region to be treated using the apparatus described in Example 2. Treatment parameters are selected so as to produce voids at a depth in the skin just below the dermal-epidermal junction of the skin, which can be between about 100 μm and about 300 μm in depth, depending on the area of the body being treated and the subject. Treatment parameters are also selected so as to produce the desired density of pigmentation following treatment. A solution containing a melanocyte stem cell, a growth factor and a scaffolding material is injected or implanted into the base of the micropore channels or voids. The stem cells attach to the scaffolding material and the epidermis, dermal-epidermal junction, or papillary dermis. The stem cells to develop into a normal melanocyte, producing melanin. Treatment with various factors, such as, for example, differentiation factors and/or proliferation factors, at various times following injection or implantation can be used to help the stem cells differentiate, populate the micropore channel or void, and produce melanin.

Those skilled in the art may devise other contamination reducing methods or devices without departing from the spirit and scope of the present invention.

In summary, the present invention is described above in terms of a preferred and other embodiments. The invention is not limited, however, to the embodiments described and depicted. Rather, the invention is limited only by the claims appended hereto.

All printed patents and publications referred to in this application are hereby incorporated herein in their entirety by this reference.

What is claimed is:

1. A method of treating or preventing loss of pigmentation in the skin of a subject in need thereof, the method comprising:
irradiating skin with laser irradiation to form a plurality of micropore channels or voids wherein the micropore channels or voids extend into the basal layer of the epidermis; and
implanting a composition into the micropore channels or voids, wherein the composition comprises at least one cell capable of producing melanin and a growth media.

2. The method of claim 1, wherein the composition is implanted 1 min after the formation of the plurality of micropore channels or voids.

3. The method of claim 1, wherein the composition is implanted 1 hr after the formation of the plurality of micropore channels or voids.

4. The method of claim 1, wherein the composition is implanted 1 day after the formation of the plurality of micropore channels or voids.

5. The method of claim 1, wherein the plurality of micropore channels or voids are elongated.

6. The method of claim 5, wherein the viable tissue separates the plurality of elongated micropore channels or voids.

7. The method of claim 1, wherein the micropore channels or voids extend into the dermal-epidermal junction of the skin.

8. The method of claim 1, wherein the micropore channels or voids extend into the dermis of the skin.

7. The method of claim 1, wherein the at least one cell is a melanocyte, a melanoblast, a stem cell, or combinations thereof.

8. The method of claim 1, wherein the at least one cell is a melanocyte.

9. The method of claim 1, wherein the at least one cell is an embryonic stem cell, fetal stem cell, umbilical cord blood stem cell, or an adult stem cell.

10. The method of claim 9, wherein the at least one cell is an adult stem cell.

11. The method of claim 10, wherein the adult stem cell is derived from adipose tissue, hair follicle, or bone marrow.

12. The method of claim 9, wherein the stem cell is a melanocyte stem cell.

13. The method of claim 1, wherein the growth media contains a compound selected from the group consisting of basic fibroblast growth factor (bFGF), bovine pituitary extract (BPE), fetal bovine serum, hydrocortisone, insulin, phenol red, phorbol myristate acetate (PMA), epidermal growth factor (EGF), transferrin, epinephrine, calcium chloride, penicillin, streptomycin, gentamycin, Amphotericin B, and combinations thereof.

14. The method of claim 1, wherein the composition further comprises a scaffolding material.

15. The method of claim 14, wherein the scaffolding material is selected from the group consisting of poly(lactic-co-glycolic acid) (PLGA), fibronectin, collagen 1, collagen 3, peptide hydrogels, and carbon nanotubes, and mixtures thereof.

16. The method of claim 14, wherein the scaffolding material is PLGA.

17. The method of claim 1, wherein the composition further comprises a differentiation factor.

18. The method of claim 17, wherein the differentiation factor is selected from the group consisting of nerve growth factor (NGF), platelet-derived growth factors (PDGF), thryotropin releasing hormone (TRH), transforming growth factor betas (TGFβs), insulin-like growth factor (IGF-1), and combinations thereof.

19. The method of claim 1, wherein the composition further comprises a proliferation factor.

20. The method of claim 19, wherein the proliferation factor is selected from the group consisting of basic fibroblast growth factor (bFGF or FGF-2), hepatocyte growth factor/scatter factor (HGF/SF), macrophage colony stimulating factor (M-CSF), endothelin-1 (ET-1), melanocyte stimulating hormone (MSH), transforming growth factor-beta (TGFβ), bovine pituitary extract (BPE), fetal bovine serum, bovine brain extract, hydrocortisone, insulin, phenol red, phorbol myristate acetate (PMA), epidermal growth factor (EGF), nerve growth factor, transferrin, epinephrine, calcium chloride, penicillin, streptomycin, gentamycin, Amphotericin B, cholera toxin, triiodothyronine, and combinations thereof.

21. The method of claim 1, wherein the composition further comprises a pigment.

22. The method of claim 21, wherein the pigment comprises a tattoo ink.

23. An apparatus for treating or preventing loss of pigmentation in the skin of a subject in need thereof, the apparatus comprising:
a handpiece movable over skin wherein the handpiece is arranged to receive an optical beam and focus the optical beam at a plurality of spaced-apart locations on the skin thereby creating a plurality of micropore channels or voids in the skin for the deposition of a composition, wherein the composition comprises at least one cell capable of producing melanin and a growth media.

24. The apparatus of claim 23, further comprising an applicator arranged to deposit a composition in the voids following the formation of the micropore channels or voids.

25. The apparatus of claim 24, wherein the applicator further comprises a removable tip that attaches to the handpiece.

26. The apparatus of claim 23, wherein the at least one cell capable of producing melanin is a melanocyte.

27. The apparatus of claim 23, wherein the at least one cell capable of producing melanin is a melanoblast.

28. The apparatus of claim 23, wherein the at least one cell capable of producing melanin is a melanocyte stem cell.

29. The apparatus of claim 23, wherein the media comprises a melanocyte growth media.

30. The apparatus of claim 29, wherein the growth media contains a compound selected from the group consisting of basic fibroblast growth factor (bFGF), bovine pituitary extract (BPE), fetal bovine serum, hydrocortisone, insulin, phenol red, phorbol myristate acetate (PMA), epidermal growth factor (EGF), transferrin, epinephrine, calcium chloride, penicillin, streptomycin, gentamycin, Amphotericin B, and combinations thereof.

31. The apparatus of claim 23, wherein the composition further comprises a scaffolding material.

32. The apparatus of claim 31, wherein the scaffolding material is selected from the group consisting of poly(lactic-co-glycolic acid) (PLGA), fibronectin, collagen 1, collagen 3, peptide hydrogels, carbon nanotubes, and combinations thereof.

33. The apparatus of claim 23, wherein the composition further comprises a differentiation factor.

34. The apparatus of claim 33, wherein the differentiation factor is selected from the group consisting of nerve growth factor (NGF), platelet-derived growth factors (PDGF), thryotropin releasing hormone (TRH), transforming growth factor betas (TGFβs), insulin-like growth factor (IGF-1), and combinations thereof.

35. The apparatus of claim 23, wherein the composition further comprises a proliferation factor.

36. The apparatus of claim 35, wherein the proliferation factor is selected from the group consisting of basic fibroblast growth factor (bFGF or FGF-2), hepatocyte growth factor/scatter factor (HGF/SF), macrophage colony stimulating factor (M-CSF), endothelin-1 (ET-1), melanocyte stimulating hormone (MSH), transforming growth factor-beta (TGFβ), bovine pituitary extract (BPE), fetal bovine serum, bovine brain extract, hydrocortisone, insulin, phenol red, phorbol myristate acetate (PMA), epidermal growth factor (EGF), nerve growth factor, transferrin, epinephrine, calcium chloride, penicillin, streptomycin, gentamycin, Amphotericin B, cholera toxin, triiodothyronine, and combinations thereof.

37. The apparatus of claim 23, wherein the composition further comprises a pigment.

38. The apparatus of claim 37, wherein the pigment comprises a tattoo ink.

39. The apparatus of claim 23, wherein viable tissue separates the plurality of voids.

40. The apparatus of claim 39, wherein the viable tissue separating any two voids is between about 50 μm and about 500 μm at its narrowest point.

41. The apparatus of claim 23, wherein the voids are elongated.

42. The apparatus of claim 23, wherein the voids are created with a density of about 200 to about 4000 voids per cm in a single pass.

43. The apparatus of claim 23, wherein the voids are created at a rate of about 10 to about 5000 voids per second.

44. The apparatus of claim 23, wherein the voids are created at a rate of about 100 to about 5000 voids per second.

45. The apparatus of claim 23, wherein the pulse energy is about 5 to about 40 mJ per void.

46. The apparatus of claim 23, further comprising a scanner.

47. The apparatus of claim 46, wherein the scanner comprises a reflective rotating scanner.

48. The apparatus of claim 46, wherein the scanner comprises one or more galvanometer scanners.

49. The apparatus of claim 23, wherein the optical beam is emitted by a laser.

50. The apparatus of claim 49, wherein the laser is a CO₂ laser with a wavelength of about 10.6 μm.

51. The apparatus of claim 23, wherein the optical beam has an absorption coefficient in water of about 100 to about 12,300 cm⁻¹.

52. The apparatus of claim 23, wherein the optical beam has an absorption coefficient in water of about 500 to about 1000 cm⁻¹.

53. The apparatus of claim 23, wherein the voids are between about 30 μm and about 500 μm in depth

54. The apparatus of claim 23, further comprising a vacuum that removes debris that is removed from the skin during creation of the voids.

55. The apparatus of claim 23, further comprising a system that creates a positive pressure in a chamber containing the composition.

56. A method of treating loss of pigmentation in the skin of a subject in need thereof, the method comprising:
irradiating skin with laser irradiation to form a plurality of micropore channels or voids, wherein the micropore channels or void extend into the basal layer of the epidermis; and
implanting a composition into the micropore channels or voids, wherein the composition comprises a pigment.

57. The method of claim 56, wherein viable tissue separates the plurality of micropore channels or voids.
